SEQUENCE LISTING (43 sequences)

SEQ ID NO: 1 - protein, 10 aa, Artificial Sequence
  Description of Artificial Sequence Synthetic peptide
KLDDLHVAMK

SEQ ID NO: 2 - protein, 11 aa, Artificial Sequence
  Description of Artificial Sequence Synthetic peptide
KNDHLSEMVEK

SEQ ID NO: 3 - protein, 11 aa, Artificial Sequence
  Description of Artificial Sequence Synthetic peptide
KQNLKLLETKH

SEQ ID NO: 4 - protein, 11 aa, Artificial Sequence
  Description of Artificial Sequence Synthetic peptide
KLLETKHITEK

SEQ ID NO: 5 - protein, 11 aa, Artificial Sequence
  Description of Artificial Sequence Synthetic peptide
KNYIHIARNLK

SEQ ID NO: 6 - protein, 12 aa, Artificial Sequence
  Description of Artificial Sequence Synthetic peptide
KKAPEDKRTQMH

SEQ ID NO: 7 - protein, 8 aa, Artificial Sequence
  Description of Artificial Sequence Synthetic peptide
KLKLKHRK

SEQ ID NO: 8 - protein, 7 aa, Artificial Sequence
  Description of Artificial Sequence Synthetic peptide
KEHYAFK

SEQ ID NO: 9 - protein, 11 aa, Artificial Sequence
  Description of Artificial Sequence Synthetic peptide
KEGVVKLKLNH

SEQ ID NO: 10 - protein, 11 aa, Artificial Sequence
  Description of Artificial Sequence Synthetic peptide
KGLVMPIACHK

SEQ ID NO: 11 - protein, 13 aa, Artificial Sequence
  Description of Artificial Sequence Synthetic peptide
KRVTDIEFKGPRH

SEQ ID NO: 12 - protein, 10 aa, Artificial Sequence
  Description of Artificial Sequence Synthetic peptide
KGPRHTSVWK

SEQ ID NO: 13 - protein, 11 aa, Artificial Sequence
  Description of Artificial Sequence Synthetic peptide
KWYHRFCDIQK

SEQ ID NO: 14 - protein, 15 aa, Artificial Sequence
  Description of Artificial Sequence Synthetic peptide
HLFTLLIKDTVDNRK

SEQ ID NO: 15 - protein, 12 aa, Artificial Sequence
  Description of Artificial Sequence Synthetic peptide
KQTITDWQKLTH

SEQ ID NO: 16 - protein, 15 aa, Artificial Sequence
  Description of Artificial Sequence Synthetic peptide
HCNNPDNDKYEPLFK

SEQ ID NO: 17 - protein, 8 aa, Artificial Sequence
  Description of Artificial Sequence Synthetic peptide
KLKHRKYK

SEQ ID NO: 18 - protein, 17 aa, Artificial Sequence
  Description of Artificial Sequence Synthetic peptide
KFNWTNVEKLTYEKNDH

SEQ ID NO: 19 - protein, 18 aa, Artificial Sequence
  Description of Artificial Sequence Synthetic peptide
HVAMKFEDLKLAIIDDPK

SEQ ID NO: 20 - protein, 9 aa, Artificial Sequence
  Description of Artificial Sequence Synthetic peptide
HLKMLYTKK

SEQ ID NO: 21 - protein, 8 aa, Artificial Sequence
  Description of Artificial Sequence Synthetic peptide
KKRDDHLK

SEQ ID NO: 22 - protein, 7 aa, Artificial Sequence
  Description of Artificial Sequence Synthetic peptide
KRDDHLK

SEQ ID NO: 23 - protein, 8 aa, Artificial Sequence
  Description of Artificial Sequence Synthetic peptide
KFHRVSTK

SEQ ID NO: 24 - protein, 9 aa, Artificial Sequence
  Description of Artificial Sequence Synthetic peptide
KIDRWFLHK

SEQ ID NO: 25 - protein, 11 aa, Artificial Sequence
  Description of Artificial Sequence Synthetic peptide
KLSHDVLLRAK

SEQ ID NO: 26 - protein, 11 aa, Artificial Sequence
  Description of Artificial Sequence Synthetic peptide
KLSHDVLLGAK

SEQ ID NO: 27 - protein, 11 aa, Artificial Sequence
  Description of Artificial Sequence Synthetic peptide
KLSHDVLLHAK

SEQ ID NO: 28 - protein, 11 aa, Artificial Sequence
  Description of Artificial Sequence Synthetic peptide
KIPRWDLGKFH

SEQ ID NO: 29 - protein, 13 aa, Artificial Sequence
  Description of Artificial Sequence Synthetic peptide
HTKLSHDVLLRAK

SEQ ID NO: 30 - protein, 10 aa, Artificial Sequence
  Description of Artificial Sequence Synthetic peptide
HAKRIGFSDK

SEQ ID NO: 31 - protein, 7 aa, Artificial Sequence
  Description of Artificial Sequence Synthetic peptide
KHNHYNK

SEQ ID NO: 32 - protein, 11 aa, Artificial Sequence
  Description of Artificial Sequence Synthetic peptide
KHYNHYNHYNK

SEQ ID NO: 33 - protein, 8 aa, Artificial Sequence
  Description of Artificial Sequence Synthetic peptide
KHYNHYNK

SEQ ID NO: 34 - protein, 9 aa, Artificial Sequence
  Description of Artificial Sequence Synthetic peptide
KHYNNYNNK

SEQ ID NO: 35 - protein, 9 aa, Artificial Sequence
  Description of Artificial Sequence Synthetic peptide
KHYNNKHYK

SEQ ID NO: 36 - protein, 8 aa, Artificial Sequence
  Description of Artificial Sequence Synthetic peptide
KLHNEKEK

SEQ ID NO: 37 - protein, 11 aa, Artificial Sequence
  Description of Artificial Sequence Synthetic peptide
HNEKEKFLQEK

SEQ ID NO: 38 - protein, 11 aa, Artificial Sequence
  Description of Artificial Sequence Synthetic peptide
KEKFLQEKTSH

SEQ ID NO: 39 - protein, 10 aa, Artificial Sequence
  Description of Artificial Sequence Synthetic peptide
KFLQEKTSHK

SEQ ID NO: 40 - protein, 23 aa, Artificial Sequence
  Description of Artificial Sequence Synthetic peptide
KLLQEKTSRKRYSRSREREQKSH

SEQ ID NO: 41 - protein, 10 aa, Artificial Sequence
  Description of Artificial Sequence Synthetic peptide
KHYNNKHYNK

SEQ ID NO: 42 - protein, 9 aa, Artificial Sequence
  Description of Artificial Sequence Synthetic peptide
KHYNKHYNK

SEQ ID NO: 43 - protein, 8 aa, Artificial Sequence
  Description of Artificial Sequence Synthetic peptide
KHYNKHYK

(19) United States
(12) Patent Application Publication
(10) Pub. No.: US 2016/0287684 A1
(43) Pub. Date: Oct. 6, 2016

(54) THERAPIES, VACCINES, AND PREDICTIVE METHODS FOR BEE COLONY COLLAPSE SYNDROME

(71) Applicants: Samuel BOGOCH, New York, NY (US); Elenore S. BOGOCH, New York, NY (US); Anne-Elenore Bogoch BORSANYI, New York, NY (US); Samuel Winston BOGOCH, Oakland, CA (US)

(72) Inventors: Samuel BOGOCH, New York, NY (US); Elenore S. BOGOCH, New York, NY (US); Anne-Elenore Bogoch BORSANYI, New York, NY (US); Samuel Winston BOGOCH, Oakland, CA (US)

(21) Appl. No.: 15/038,389

(22) PCT Filed: Nov. 21, 2014
(86) PCT No.: PCT/US2014/066742

§ 371 Date: May 20, 2016

Related U.S. Application Data
(60) Provisional application No. 61/907,802, filed on Nov. 22, 2013.

(51) Int. Cl. A61K 39/00; C07K 14/37; G01N 33/68; C07K 16/18; C12N 9/14; C07K 14/435; C07K 16/14

(52) U.S. Cl. CPC A61K 39/0003; C07K 14/43531; C07K 14/37; C07K 14/43572; A61K 39/0002; C07K 16/14; C07K 16/18; C12N 9/14; C12Y 306/01003; G01N 33/6818; A61K 2039/552; C07K 2317/34

(57) ABSTRACT

The present invention provides therapies, vaccines, and predictive methods for Colony Collapse Disorder in bees and provides compounds for diagnosing, preventing, and treating Colony Collapse Disorder, A first non-limiting aspect of the present invention provides an isolated or synthesized peptide of up to 100 amino acid residues comprising at least one peptide sequence that is at least 70%, 80%, 90%, or 95% homologous with at least one Replikin peptide sequence identified in a CCD factor in honeybees or in any honeybee including but not limited to at least one Replikin sequence identified in at least one isolate of Varroa destructor, Nosema species, Deformed wing virus, Israeli acute paralysis virus, Kashmir bee virus. Sac brood virus, or in honeybees, including, but not limited to Apis mellifera and Apis cerana, or any Replikin sequence in the ATPase or complementary sex-determination (csd) gene of a honeybee.

This application claims priority to U.S. Provisional Application Ser. No. 61/907,802, filed Nov. 22, 2014, which is herein incorporated by reference in its entirety.

### SEQUENCE LISTING

The instant application contains a Sequence Listing, which has been submitted electronically in ASCII format and is hereby incorporated by reference in its entirety. Said ASCII copy, created on Nov. 20, 2014, is named 13795-48976 SL.txt and is 10,751 bytes in size.

### FIELD OF THE INVENTION

The present invention relates to therapies for preventing and treating Bee Colony Collapse Syndrome (CCD), methods of predicting and identifying outbreaks of CCD, and compounds for diagnostic, therapeutic, and/or preventive purposes in CCD.

### BACKGROUND OF THE INVENTION

Colony collapse disorder (CCD) is a disorder observed in honeybees where the worker bees of a colony disappear with suddenness. Honeybees include and are not limited to Apis cerana (Asiatic honeybee) and Apis mellifera (western or European honeybee). The disorder was first reported around 2006 by commercial beekeepers who observed adult worker bees fleeing their hive and dying. Prior to observation of CCD, colony loss of around 15% was not abnormal. Since 2006, however, mortality in commercial operations has been calculated at up to ⅓ of bees and hives (28% to 33%).

Until now, reasons for an increase in CCD have not been clear with many culprits having been suggested, including, neonicotinoid-class pesticides, infections with Varroa and Acarapis mites, loss of bee habitat, nutritional deficiencies, electronic communications, and a combination of certain of these factors. Id. The second annual report of the U.S. Colony Collapse Disorder Steering Committee reported in 2010 that many associations between environmental and pathogenic factors and CCD have been identified. Id. These factors include pesticides, parasites, and pathogens. Nevertheless, the report noted “it is becoming increasingly clear that no single factor alone is responsible . . . .” Colony Collapse Disorder Progress Report, USDA June 2010.

One pathogen that has been associated with CCD is the parasitic mite classified as Varroa destructor. Varroa destructor is an external parasitic mite of honeybees, including Apis cerana (Asiatic honeybee) and Apis mellifera (western or European honeybee). The mites are specific to honeybee colonies and weaken bees by feeding on hemolymph (analog of blood). The mites often spread pathogenic viruses, including, for example, deformed wing virus.

Another pathogen that has been associated with CCD is the genus of fungi classified as Nosema. Nosema spp. are microsporidians, which are unicellular parasites currently classified as fungi. Nosema species include and are not limited to Nosema apis (which is understood to be the most common of honeybee diseases) and Nosema ceranae (which is understood to parasitize Asiatic honeybees).

Many viruses have been associated with CCD. Examples include the Deformed Wing virus, the Israel acute paralysis virus, the Kashmir bee virus, and the Sacbrood virus. The Deformed wing virus (DWV) is an RNA virus. It has been observed worldwide in honeybees. The virus is often transmitted by Varroa destructor. Three structural proteins have been identified in the virus including VP1, VP2, and VP3. The genome also contains an RNA helicase, a chymotrypsin-like 3C protease, and an RNA-dependent RNA polymerase. There is also an unconfirmed VP4 gene. The viral genome was published at Lanzi G. et al. (2006) Molecular and biological characterization of deformed wing virus of honeybees (Apis mellifera L.). J Virol. 2006 80(10):4998-5009. The Israeli acute paralysis virus (IAPV) is a single stranded RNA positive-strand virus of Aparavirus. See NCBI, Israeli acute paralysis virus. It has also been associated with CCD. The Kashmir bee virus (KBV) is likewise associated with CCD. See NCBI, Kashmir bee virus. It is also a single-stranded RNA positive-strand virus of Aparavirus. The Sacbrood virus (SBV) causes the disease Sacbrood, which is manifest in larvae that fail to pupate. SBV is understood to be picornavirus-like positive-stranded RNA viruses. Grabensteiner et al., “Sacbrood Virus of the Honeybee (Apis mellifera): Rapid Identification and Phylogenetic Analysis Using Reverse Transcription-PCR,” Clin Diagn Lab Immunol. 2001 January; 8(1): 93-104. SBV has likewise been associated with CCD.

There is a need in the art for methods of quantitatively identifying factors causing CCD. There is likewise a need in the art for therapies and prophylactics against CCD. See, e.g., Engelsdorp et al., Bee Informed Team, Winter Loss Survey 2012-2013, Walsh, B., “Honeybees are still dying and we still don't know why,” Time, Science and Space May 7, 2013, Goulson, D., “We have no Plan Bee for when we run out of pollinators,” Financial Times (Nov. 9 and 10, 2013) p. 9, Spleen et al., J. Agriculture Research 52(2):44-53, 2013, Hildebrandt, A., “Huge honey bee losses across Canada dash hopes of upturn, Manitoba, New Brunswick and Ontario suffer highest death rates,” CBC News Posted: Sep. 11, 2013 5:21 AM ET Last Updated: Sep. 11, 2013 5:44 AM ET.

Replikin peptides are a family of small peptides that have been correlated with the phenomenon of rapid replication in SARS, influenza, malaria, West Nile virus, foot and mouth disease, and many other pathogens. See, e.g., WO 2008/143717. Replikin peptides have likewise been generally correlated with the phenomenon of rapid replication in viruses, organisms, and malignancies. Identification of Replikin peptides has provided targets for detection and treatment of pathogens, including vaccine development against virulent pathogens such as influenza virus, malaria, West Nile virus, and foot and mouth disease virus. See, e.g., WO 2008/143717. In general, knowledge of and identification of this family of peptides enables development of effective therapies and vaccines for any pathogen that harbors Replikin sequences. The phenomenon of the association of Replikin sequences with rapid replication and virulence has been fully described in U.S. Pat. No. 7,189,800; U.S. Pat. No. 7,176,275; U.S. Pat. No. 7,442,761; U.S. Pat. No. 7,894,999, U.S. Pat. No. 8,050,871, and WO 2008/156914. Both Replikin concentration (number of Replikin sequences per 100 amino acids) and Replikin composition have been correlated with the functional phenomenon of rapid replication.

In response to the continuing need to understand the causes of CCD and the continuing need for therapies against CCD and despite extensive efforts of others to understand CCD, applicants have now surprisingly applied their previous discovery of Replikin chemistry in the virus genome structure to methods of identifying factors causing CCD and therapies against the factors causing CCD. They have surprisingly identified conserved targets in CCD against which vaccines are provided and likewise may be provided prior to or at the outset of any further colony disorder. Such vaccine development can be undertaken in as few as seven days.

### SUMMARY OF THE INVENTION

The present invention provides compounds for diagnostic, therapeutic, and/or preventive purposes against CCD and methods of predicting and diagnosing outbreaks of CCD.

A first non-limiting aspect of the present invention provides an isolated or synthesized peptide of up to 100 amino acid residues comprising at least one peptide sequence that is at least 70%, 80%, 90%, or 95% homologous with at least one Replikin peptide sequence identified in a CCD factor in honeybees or in any honeybee including but not limited to at least one Replikin sequence identified in at least one isolate of Varroa destructor, Nosema species, Deformed wing virus, Israeli acute paralysis virus, Kashmir bee virus, Sacbrood virus, or in honeybees, including, but not limited to Apis mellifera and Apis cerana, or any Replikin sequence in the ATPase or complementary sex-determination (csd) gene of a honeybee.

In a non-limiting embodiment, the isolated or synthesized peptide may consist essentially of at least one Replikin peptide sequence identified in a CCD factor in honeybees or at least one homologue of said at least one Replikin peptide sequence identified in a CCD factor in honeybees. The homologue of said at least one Replikin peptide sequence may be 50%, 60%, 70%, 80%, 90%, or 95% or more homologous with said Replikin peptide sequence. The isolated or synthesized peptide may consist of at least one Replikin peptide sequence identified in a CCD factor or at least one homologue of said at least one Replikin peptide sequence identified in a CCD factor. Another non-limiting embodiment provides an isolated or synthesized peptide sequence comprising at least one functional fragment of a Replikin sequence identified in a CCD factor. In a non-limiting embodiment, the protein fragment or peptide may be isolated or derived from one of the gene segments of the genome of a CCD factor, including from an isolate of Varroa destructor, Nosema species, Deformed wing virus, Israeli acute paralysis virus, Kashmir bee virus, Sacbrood virus, or in honeybees, including, and not limited to Apis mellifera, Apis cerana, Apis dorsata (giant honeybee), Euglossa hemichlora (orchid bee), Bombus impatiens (common eastern bumble bee), Bombus terrestris (buff-tailed bumblebee), Apis florea (little honeybee), Megachile rotundata (alfalfa leafcutting bee), or any Replikin sequence in the ATPase or csd gene of a honeybee or related bee. The gene segment of Deformed wing virus may be VP1, VP2, VP3, VP4, RNA helicase, a chymotrypsin-like 3C protease, or an RNA-dependent RNA polymerase.

In a non-limiting embodiment, the isolated or synthesized peptide may comprise at least one Replikin sequence of SEQ ID NO(s): 1-19, 20-22, 23-30, or 31-43 or at least one homologue of SEQ ID NO(s): 1-19, 20-22, 23-30, or 31-43. In a non-limiting embodiment, the homologue may be 50%, 60%, 70%, 80%, 90%, or 95% or more homologous with said Replikin sequence. The isolated or synthesized peptide may consists essentially of at least one Replikin sequence of SEQ ID NO(s): 1-19, 20-22, 23-30, or 31-43, or at least one homologue of SEQ ID NO(s): 1-19, 20-22, 23-30, or 31-43. In another non-limiting embodiment, the isolated or synthesized peptide may consist of at least one Replikin sequence of SEQ ID NO(s): 1-19, 20-22, 23-30, or 31-43 or at least one homologue of SEQ ID NO(s): 1-19, 20-22, 23-30, or 31-43. In a non-limiting embodiment, the homologue may be 50%, 60%, 70%, 80%, 90%, or 95% or more homologous with said Replikin sequence. Another non-limiting embodiment provides an isolated or synthesized peptide sequence comprising at least one functional fragment of at least one Replikin peptide sequence of SEQ ID NO(s): 1-43. In a further non-limiting embodiment of the first aspect, the isolated or synthesized peptide consists of up to 10, 20, 30, 40, 50, 60, 70, 80, or 90 amino acid residues. Another non-limiting embodiment of the first aspect of the invention provides a biosynthetic composition comprising the peptide of an aspect of the invention. In a further non-limiting embodiment, the biosynthetic composition consists essentially of a Replikin peptide of a CCD factor or consists of a Replikin peptide of a CCD factor. In a non-limiting embodiment, an isolated peptide is chemically synthesized by solid phase methods.

A second non-limiting aspect of the present invention provides an immunogenic and/or blocking composition comprising at least one peptide of any one of the above-listed peptides including and not limited to an isolated or synthesized peptide of up to 100 amino acid residues comprising at least one Replikin peptide sequence identified in a CCD factor or at least one homologue of said at least one Replikin peptide identified in a CCD factor or at least one functional fragment of at least one Replikin peptide sequence identified in a CCD factor. In a non-limiting embodiment of the second aspect of the present invention, the immunogenic and/or blocking composition comprises at least one peptide sequence of SEQ ID NO(s): 1-43. In a further non-limiting embodiment, the immunogenic and/or blocking composition comprises at least one peptide consisting essentially of any one of SEQ ID NO(s): 1-43. In further non-limiting embodiment, the immunogenic and/or blocking composition comprises at least one peptide consisting of any one of SEQ ID NO(s): 1-43 or at least one functional fragment of any one of SEQ ID NO(s): 1-43.

In a non-limiting embodiment, the immunogenic and/or blocking composition comprises a mixture of isolated or chemically-synthesized peptides of each of SEQ ID NO(s): 1-19, SEQ ID NO(s): 20-22, SEQ ID NO(s): 23-30, or SEQ ID NO(s): 31-43. In a non-limiting embodiment, the composition comprises each of the isolated or chemically-synthesized peptides of SEQ ID NO(s): 1-43. In a non-limiting embodiment, the composition comprises an approximately equal molar mixture of the isolated or synthesized peptides of SEQ ID NO(s): 1-19, 20-22, 23-30, or 31-43. In a non-limiting embodiment, the mixture comprises approximately equal weight of the isolated or synthesized peptides of SEQ ID NO(s): 1-19, 20-22, 23-30, or 31-43.

A third non-limiting aspect of the present invention provides a vaccine comprising at least one peptide of any one of the above-listed peptides. The at least one peptide may include and is not limited to at least one isolated or chemically-synthesized peptide of up to 100 amino acid residues comprising at least one peptide sequence that is at least 70%, 80%, 90%, or 95% homologous with at least one Replikin peptide sequence identified in a CCD factor in honeybees or in any honeybee or at least one isolated or synthesized peptide of up to 100 amino acid residues comprising at least one Replikin peptide sequence identified in a CCD factor in honeybees or in any honeybee. In a non-limiting embodiment of the third aspect of the present invention, the vaccine comprises at least one isolated or chemically-synthesized homologue of SEQ ID NO(s): 1-43 that is at least 80% homologous with at least one of SEQ ID NO(s): 1-43, at least one peptide sequence of any one of SEQ ID NO(s): 1-43, at least one peptide sequence consisting essentially of any one of SEQ ID NO(s): 1-43, at least one peptide sequence consisting of any one of SEQ ID NO(s): 1-43, at least one functional fragment of any one of SEQ ID NO(s): 1-43, and/or at least one functional fragment of a Replikin peptide sequence identified in a CCD factor.

In a further non-limiting embodiment of the third aspect, the vaccine comprises a mixture of at least two isolated or chemically-synthesized peptide sequences of any of SEQ ID NO(s): 1-43 and/or a mixture of at least two isolated or chemically-synthesized homologues of peptide sequences of any of SEQ ID NO(s): 1-43. In a further non-limiting embodiment, the vaccine comprises a mixture of a plurality of peptide sequences consisting essentially of any one or more of SEQ ID NO(s): 1-43. In a further non-limiting embodiment, the vaccine comprises a mixture of a plurality of peptide sequences consisting of any one or more of SEQ ID NO(s): 1-43. In a further non-limiting embodiment, the vaccine comprises a mixture of isolated or chemically-synthesized peptide sequences consisting of each of SEQ ID NO(s): 1-19, 20-22, 23-30, or 31-43. In a further non-limiting embodiment, the vaccine comprises a mixture of a plurality of isolated or chemically-synthesized peptides consisting of each of SEQ ID NO(s): 1-43.

In another non-limiting embodiment of the third aspect of the invention, the vaccine comprises a mixture of Replikin peptides. In a non-limiting embodiment, the vaccine comprises an approximately equal molar mixture of the isolated or synthesized peptides of SEQ ID NO(s): 1-19, 20-22, 23-30, 31-43, or 1-43. In a further non-limiting embodiment, the vaccine comprises approximately equal weight of the isolated or synthesized peptides of SEQ ID NO(s): 1-19, 20-22, 23-30, 31-43, or 1-43.

In a further non-limiting embodiment, the vaccine comprises a pharmaceutically-acceptable carrier and/or adjuvant. In a further non-limiting embodiment, the vaccine is for the treatment or prevention of CCD or an infection or infestation of a CCD factor.

A fourth non-limiting aspect of the invention provides a binding agent that binds to at least a portion of an amino acid sequence of at least one peptide sequence that is 30%, 40%, 50%, 60%, 70%, 80%, 90%, or 95% or more homologous with at least one Replikin peptide sequence identified in a CCD factor. In non-limiting embodiment, the binding agent binds at least a portion of at least one Replikin peptide sequence identified in an isolate of Varroa destructor, Nosema species, Deformed wing virus, Israeli acute paralysis virus, Kashmir bee virus, Sacbrood virus, or in the ATPase or complementary sex-determination gene of a honeybee or at least one homologue of said at least one Replikin peptide sequence that is at least 80% homologous with said at least one Replikin peptide sequence. In a further embodiment, the at least one Replikin peptide sequence identified in a CCD factor is at least one peptide sequence of SEQ ID NO(s): 1-43. The binding agent may be, and is not limited to, an antibody, antibody fragment, or any other binding agent. The binding agent may be isolated or chemically-synthesized.

A fifth non-limiting aspect of the present invention provides a method of making a vaccine comprising: selecting at least one isolated or chemically-synthesized peptide comprising at least one sequence that is at least 30%, 40%, 50%, 60%, 70%, 80%, 90% or 95%, or 100% homologous with at least one Replikin peptide sequence identified in a CCD factor or any honeybee as a component of a vaccine; and making said vaccine comprising said at least one isolated or chemically-synthesized peptide. In a non-limiting embodiment, the method of making a vaccine comprises selecting at least one isolated or synthesized peptide of SEQ ID NO(s): 1-43 as at least one component and making said vaccine with the at least one component.

In another non-limiting embodiment, the method of making a vaccine comprises selecting at least two, three, four, five, six, seven, eight, nine, ten, eleven, twelve, thirteen, fourteen, fifteen, sixteen, seventeen, eighteen, nineteen, or up to forty-three or more isolated or synthesized Replikin peptide sequences identified in a CCD factor or any honeybee and/or isolated or synthesized functional fragments of Replikin peptide sequences identified in a CCD factor or any honeybee. In a further embodiment, the isolated or synthesized Replikin peptide sequences or functional fragments of Replikin peptide sequences identified in a CCD factor comprise at least one peptide sequence of SEQ ID NO(s): 1-43, at least one homologue of at least one peptide sequence of SEQ ID NO(s): 1-43, or at least one functional fragment of at least one Replikin peptide sequence identified in a CCD factor. In another non-limiting embodiment, the at least one isolated or synthesized peptide has the same amino acid sequence as at least one peptide identified in a relatively lethal strain of a CCD factor up to seven days, one month, six months, one year, two years, or three years prior to making said vaccine.

A sixth non-limiting aspect of the present invention provides a method for preventing or treating CCD or an infestation or infection of a honeybee from a CCD factor comprising administering at least one isolated or synthesized peptide consisting of up to 100 amino acid residues comprising at least one peptide sequence to a honeybee where the peptide sequence is at least 30%, 40%, 50%, 60%, 70%, 80%, 90% or 95%, or 100%, homologous with at least one Replikin peptide sequence identified in a CCD factor or any honeybee. In a further non-limiting embodiment, the Replikin peptide sequence is at least one peptide sequence of SEQ ID NO(s): 1-43. In a non-limiting embodiment, the at least one isolated or synthesized peptide consists of at least one peptide sequence that is at least 30%, 40%, 50%, 60%, 70%, 80%, 90%, or 95% or more homologous with at least one of the peptide sequences of SEQ ID NO(s): 1-43. In another non-limiting embodiment, the at least one isolated or synthesized peptide of SEQ ID NO(s): 1-43 is administered to a honeybee. In a further non-limiting embodiment the at least one Replikin peptide sequence is at least one peptide sequence of SEQ ID NO(s): 1-43. In a non-limiting embodiment, the at least one isolated or synthesized peptide of SEQ ID NO(s): 1-43 is administered to the honeybee orally using a sweetened delivery system such as water. Another non-limiting embodiment provides use of at least one isolated or synthesized peptide of the invention in the manufacture of an immunogenic and/or blocking composition for limiting, preventing, or reducing incidence of CCD.

A seventh non-limiting aspect of the present invention provides a method of predicting expansion or retraction of CCD comprising, identifying an increase in the percentage of isolates of at least one CCD factor having a Replikin concentration (number of Replikin sequences per 100 amino acid residues) greater than 4.0 between two time periods or identifying a decrease in the percentage of isolates of at least one CCD factor having a Replikin concentration (number of Replikin sequences per 100 amino acid residues) greater than 4.0 between two time periods. In a non-limiting embodiment, the more than two time periods are compared and the percentage of isolates in the more than two time periods shows a pattern of increase or decrease.

A non-limiting embodiment of the seventh aspect of the present invention provides a method of differentiating between relatively more lethal and relatively less lethal forms of a CCD factor. A first non-limiting embodiment provides a method of identifying and/or diagnosing a relatively more lethal form of a CCD factor comprising determining the Replikin concentration of at least one portion of at least one protein of at least one isolate of a CCD factor or at least one portion of at least one gene that expresses at least one protein of the at least one isolate of a CCD factor and comparing the Replikin concentration of the at least one isolate of a CCD factor to a comparable Replikin concentration in at least one other isolate of a CCD factor. In a further non-limiting embodiment, the at least one portion of at least one protein comprises the entirety of at least one protein expressed in a CCD factor and the comparable Replikin concentration is the Replikin concentration of the entirety of the same protein expressed in a CCD factor from the at least one other isolate of a CCD factor. In a non-limiting embodiment, the Replikin concentration of the at least one isolate of a CCD factor is a mean of Replikin concentrations determined in a plurality of isolates of a CCD factor. In a further non-limiting embodiment, the Replikin concentration of the at least one other isolate of a CCD factor is a mean of Replikin concentrations determined in a plurality of other isolates of a CCD factor. In a further non-limiting embodiment, the plurality of isolates of a CCD factor is a collection of isolates isolated in a given year and the plurality of other isolates of a CCD factor is a collection of isolates isolated in a different year. In a further non-limiting embodiment, the Replikin concentration of the more lethal isolate of a CCD factor is 3.0 or greater, 4.0 or greater, or 5.0 or greater per 100 amino acid residues. In a further non-limiting embodiment, the Replikin concentration of the more lethal isolate of a CCD factor is 4.0 or greater per 100 amino acid residues. In a further non-limiting embodiment, the Replikin concentration of the more lethal isolate of a CCD factor is 4.6 per 100 amino acid residues or greater. In a non-limiting embodiment, the CCD factor is Varroa destructor, Nosema species, Deformed wing virus, Israeli acute paralysis virus, Kashmir bee virus, or Sacbrood virus, or any other factor of CCD.

In a further non-limiting embodiment of the seventh aspect of the present invention, the at least one portion of at least one gene expressing at least one protein is at least one portion of the VP1, VP2, VP3, or VP4 gene region of Deformed wing virus.

In a further non-limiting embodiment of the seventh aspect of the present invention, the Replikin concentration of the at least one isolate of a CCD factor is greater than the Replikin concentration of the at least one other isolate of a CCD factor. In a further non-limiting embodiment the Replikin concentration is a mean Replikin concentration of a plurality of isolates with standard deviation from the mean and the standard deviation from the mean is greater than the standard deviation from the mean Replikin concentration of a plurality of other isolates.

Another non-limiting embodiment of the seventh aspect of the invention provides a method of determining an increased probability of an increase of CCD within about one year following an increase in Replikin concentration in an isolate of a CCD factor comprising identifying an increase in the concentration of Replikin sequences in at least one first isolate of a CCD factor as compared to at least one other isolate of a CCD factor wherein said at least one first isolate is isolated at a later time period than said one other isolate and wherein said increase in the concentration of Replikin sequences signifies the increased probability of the outbreak of a CCD factor within about one year following the increase in the concentration of Replikin sequences. In a non-limiting embodiment, the at least one first isolate of a CCD factor is the mean of a plurality of isolates of a CCD isolated in a given year and the at least one other isolate of a CCD factor is the mean of a plurality of isolates of a CCD factor in at least one other year.

In a non-limiting embodiment, a method of prediction comprises: (1) obtaining a plurality of isolates of a CCD factor wherein at least one of said isolates is isolated about six months to about 3 years later than at least one other of said isolates; (2) analyzing the amino acid sequence of at least one protein or protein fragment in each isolate of the plurality of isolates for the presence and concentration of Replikin sequences; (3) comparing the concentrations of Replikin sequences in the at least one protein or protein fragment in each isolate of the plurality of isolates one to another; (4) identifying an increase in the concentration of Replikin sequences in said plurality of isolates over at least one time period of about six months or greater; and (5) predicting an increase in CCD within about one month to about three years following said identified increase in the concentration of Replikin sequences. In another embodiment of the invention, the increase in CCD is predicted within about six months. In a further embodiment of the invention, the increase in CCD is predicted within about one year to about three years. In a further non-limiting embodiment, the method of prediction further comprises processing at least one step of the method on a computer.

In a further non-limiting embodiment of the seventh aspect of the invention, the method of prediction further comprises comparison of the standard deviation from the mean of Replikin concentrations of isolates of at least one CCD factor from a given time period, such as a given month, a given year, or any other given time period to another time period. In a further non-limiting embodiment, the Replikin concentration is a mean Replikin concentration of a plurality of isolates with standard deviation from the mean and the standard deviation from the mean is greater than the standard deviation from the mean Replikin concentration of a plurality of other isolates.

Another non-limiting embodiment of the seventh aspect of the invention provides a method of determining an increased probability of increased CCD incidence within about six months to about three years following an increase in Replikin concentration in isolates of multiple CCD factors comprising identifying an increase in the concentration of Replikin sequences in a plurality of first isolates of CCD factors as compared to at least one other isolate of the same or other CCD factors wherein said plurality of first isolates is isolated earlier in time than said at least one other isolate is isolated, and wherein said increase in the concentration of Replikin sequences signifies the increased probability of increased incidence of CCD within about six months to about three years following the increase in the concentration of Replikin sequences. In a non-limiting embodiment, the plurality of isolates of CCD factors are isolated at least about six months earlier than the at least one other isolate.

A further non-limiting embodiment provides a computer readable medium having stored thereon instructions which, when executed, cause a processor to perform a method of predicting an expansion of a strain of a CCD factor or an increase in virulence, morbidity, and/or mortality of a CCD factor, an increased probability of an increase in CCD, or a method of differentiating between relatively more lethal and relatively less lethal forms of a CCD factor. In a further embodiment, the processor reports a prediction to a display, user, researcher, or other machine or person. In a further embodiment, the processor identifies to a display, user, researcher, or other machine or person, a portion of a pathogen predicted to be an expanding CCD factor or predicted to increase in virulence, morbidity, and/or mortality, wherein said portion may be employed as a therapeutic or diagnostic compound. Said portion may be a Replikin peptide or plurality of Replikin peptides or any other structure or portion of said genome of said pathogen including a Replikin Peak Gene. A non-limiting computer readable medium may be non-transitory. Software comprising methods of the invention and related data may be carried on a signal.

Another non-limiting embodiment of the seventh aspect of the invention provides a computer system, including a processor coupled to a network and a memory coupled to the processor, the memory containing a plurality of instructions to perform a method of predicting an increase in CCD.

Another non-limiting embodiment of the seventh aspect of the invention provides a machine-readable storage medium having stored thereon, executable instructions that, when executed by a processor, cause the processor to provide sufficient data to a user, a display, or a printout such that said user or a user of said display or said printout may predict the lethality of CCD. Another non-limiting embodiment provides a computer system, comprising: a processor coupled to a network; a memory coupled to the processor, the memory containing a plurality of instructions to perform the method of predicting the lethality of CCD based on the regression analysis.

### BRIEF DESCRIPTION OF THE DRAWINGS

FIG. 1 illustrates annual mean Replikin concentration (with standard deviation) of amino acid sequences from isolates of Varroa destructor. The graph reflects analysis of the genomic or proteomic information publicly available for isolates at the website of the National Center for Biotechnology Information (NCBI). Data are illustrated annually from 1982 through 2012 for years in which isolates were published Annual mean Replikin concentration (Replikin sequences per 100 amino acid residues) is illustrated with black columns. Standard deviation of the mean is illustrated in gray columns with caps. The data reflect cycles in Replikin concentration in Varroa destructor. Peaks are observable in 2005, 2007, and 2011. The peaks in 2007 and 2011 coincide with high levels of Colony Collapse Disorder in North America and Europe. The peaks in 2007 and 2011 are surprisingly observed to be incidental with similar peaks in Replikin concentration in Nosema infections in honeybees, viral infections in honeybees, and Replikin concentrations in ATPase in honeybees.

FIG. 2 illustrates percent of publicly available genomic sequences of isolates of Varroa destructor for given time periods analyzed as having Replikin concentrations (Replikin sequences per 100 amino acid residues) of greater than 4.0. The graph reflects analysis of the genomic or proteomic information publicly available for isolates at the website of NCBI. Data are illustrated from 2002 through November of 2013. The data reflect a large spike in 2011 in the percent of isolates having a Replikin concentration of greater than 4.0. This large spike is coincident with high levels of Colony Collapse Disorder in North America and Europe and coincident with a similar 2011 peak in Nosema spp.

FIG. 3 illustrates annual mean Replikin concentration (with standard deviation) of amino acid sequences from isolates of Nosema spp. The graph reflects analysis of the genomic or proteomic information publicly available for isolates at the website of NCBI. Data are illustrated annually from 2008 through November 2013. Annual mean Replikin concentration (Replikin sequences per 100 amino acid residues) is illustrated with black columns. Standard deviation of the mean is illustrated in gray columns with caps. The data reflect cycles in Replikin concentration in Nosema spp. Peaks are observable in 2008 and 2011. The peaks in 2008 and 2011 coincide with high levels of Colony Collapse Disorder in North America and Europe. The peaks in 2008 and 2011 are surprisingly observed to be incidental with similar peaks in Replikin concentration in Varroa destructor infestations in honeybees, viral infections in honeybees, and Replikin concentrations in ATPase in honeybees.

FIG. 4 illustrates percent of publicly available genomic sequences of isolates of Nosema spp. for given time periods analyzed as having Replikin concentrations (Replikin sequences per 100 amino acid residues) of greater than 4.0. The graph reflects analysis of the genomic or proteomic information publicly available for isolates at the website of NCBI. Data are illustrated from 2008 through November of 2013. The data reflect a peak in 2011 in the percent of isolates having a Replikin concentration of greater than 4.0. This peak is coincident with high levels of Colony Collapse Disorder in North America and Europe.

FIG. 5 illustrates annual mean Replikin concentration (with standard deviation) of amino acid sequences from isolates of Deformed wing virus in honeybees. The graph reflects analysis of the genomic or proteomic information publicly available for isolates at the website of NCBI. Data are illustrated annually from 2002 through 2008. Annual mean Replikin concentration (Replikin sequences per 100 amino acid residues) is illustrated with black columns. Standard deviation of the mean is illustrated in gray columns with caps. The data reflect a cycle in Replikin concentration in Deformed wing virus in honeybees. Peaks are observable in 2002 and 2008. The peak in 2008 coincides with high levels of Colony Collapse Disorder in North America and Europe. The peak in 2008 is surprisingly observed to be incidental with a similar peak in Replikin concentration in Varroa destructor infestations in honeybees, Nosema infections in honeybees, other viral infections in honeybees, and Replikin concentrations in ATPase in honeybees.

FIG. 6 illustrates annual mean Replikin concentration (with standard deviation) of amino acid sequences from isolates of Israeli acute paralysis virus in honeybees. The graph reflects analysis of the genomic or proteomic information publicly available for isolates at the website of NCBI. Data are illustrated annually from 2004 through 2012. Annual mean Replikin concentration (Replikin sequences per 100 amino acid residues) is illustrated with black columns. Standard deviation of the mean is illustrated in gray columns with caps. The data reflect a cycle in Replikin concentration in Israeli acute paralysis virus in honeybees. Peaks are observable in 2004 and 2012. The peak in 2012 coincides with high levels of Colony Collapse Disorder in North America and Europe in 2013 and is incidental with 2011 peaks in Replikin concentration in Varroa destructor infestations in honeybees, Nosema infections in honeybees, other viral infections in honeybees, and Replikin concentrations in ATPase in honeybees.

FIG. 7 illustrates annual mean Replikin concentration (with standard deviation) of amino acid sequences from isolates of Kashmir bee virus in honeybees. The graph reflects analysis of the genomic or proteomic information publicly available for isolates at the website of NCBI. Data are illustrated annually from 1999 through 2012. Annual mean Replikin concentration (Replikin sequences per 100 amino acid residues) is illustrated with black columns. Standard deviation of the mean is illustrated in gray columns with caps. The data reflect a cycle in Replikin concentration in Kashmir bee virus in honeybees. A peak is observable in 2006.

FIG. 8 illustrates annual mean Replikin concentration (with standard deviation) of amino acid sequences from isolates of Sacbrood virus in honeybees. The graph reflects analysis of the genomic or proteomic information publicly available for isolates at the website of NCBI. Data are illustrated annually from 1982 through 2012. Annual mean Replikin concentration (Replikin sequences per 100 amino acid residues) is illustrated with black columns. Standard deviation of the mean is illustrated in gray columns with caps. The data reflect a cycle in Replikin concentration in Kashmir bee virus in honeybees. Peaks are observable in 2004, 2009, and 2011.

FIG. 9 illustrates annual mean Replikin concentration (with standard deviation) of amino acid sequences from isolates of all viruses in honeybees analyzed by Applicants. The graph reflects analysis of the genomic or proteomic information publicly available for isolates at the website of NCBI. Data are illustrated annually from 1982 through 2012. Annual mean Replikin concentration (Replikin sequences per 100 amino acid residues) is illustrated with black columns. Standard deviation of the mean is illustrated in gray columns with caps. The data reflect cycles in Replikin concentration in viruses in honeybees. Peaks are observable in 2004, 2009, and 2011. The peak in 2011 coincides with high levels of Colony Collapse Disorder in North America and Europe in 2013 and is incidental with 2011 peaks in Replikin concentration in Varroa destructor infestations in honeybees, Nosema infections in honeybees, other specific viruses in honeybees, and Replikin concentrations in ATPase in honeybees.

FIG. 10 illustrates percent of publicly available genomic sequences of isolates of viruses in honeybees analyzed by applicants for given time periods having Replikin concentrations (Replikin sequences per 100 amino acid residues) of greater than 4.0. The graph reflects analysis of the genomic or proteomic information publicly available for isolates at the website of NCBI. Data are illustrated from 1990 through 2012. The data reflect peaks in the percent of isolates having a Replikin concentration of greater than 4.0 in 2005 and 2011. The peak in 2011 is coincident with high levels of Colony Collapse Disorder in North America and Europe and coincident with similar peaks in Varroa destructor, Nosema spp., and ATPase in honeybees.

FIG. 11 illustrates annual mean Replikin concentration (with standard deviation) of amino acid sequences from ATPase in honeybees analyzed by Applicants. The graph reflects analysis of the genomic or proteomic information publicly available for isolates at the website of NCBI. Data are illustrated annually from 1993 through 2012. Annual mean Replikin concentration (Replikin sequences per 100 amino acid residues) is illustrated with black columns. Standard deviation of the mean is illustrated in gray columns with caps. The data reflect cycles in Replikin concentration in ATPase in honeybees. Peaks are observable in 2005, 2009, and 2011-2012. The peak in 2011 coincides with high levels of Colony Collapse Disorder in North America and Europe in 2013 and is incidental with 2011 peaks in Replikin concentration in Varroa destructor infestations in honeybees, Nosema infections in honeybees, other specific viruses in honeybees, and Replikin concentrations in ATPase in honeybees. The peak in 2009 is coincidental with Replikin concentrations in viruses in honeybees analyzed by applicants.

FIG. 12 illustrates percent of publicly available genomic sequences of isolates of ATPase in honeybees analyzed by applicants for given time periods having Replikin concentrations (Replikin sequences per 100 amino acid residues) of greater than 4.0. The graph reflects analysis of the genomic or proteomic information publicly available for isolates at the website of NCBI. Data are illustrated from 2004 through 2012. The data reflect peaks in the percent of isolates having a Replikin concentration of greater than 4.0 in 2006 and 2011-2012. The peak in 2011 is coincident with high levels of Colony Collapse Disorder in North America and Europe and coincident with similar peaks in Varroa destructor, Nosema spp., and ATPase in honeybees. The peak in 2006 is coincident with a similar peak in Kashmir bee virus and is close in time with peaks in 2004 and 2005 in Varroa destructor, Nosema, spp., and viruses in honeybees.

FIG. 13 illustrates annual percent loss of honeybee colonies in the U.S. for the given years. Data are illustrated from 2006 through November of 2013. The data reflect a peak in losses in 2008 and in 2013.

FIG. 14 illustrates annual mean Replikin concentration (with standard deviation) of amino acid sequences from complementary sex-determination (csd) gene in honeybees analyzed by Applicants. The graph reflects analysis of the genomic or proteomic information publicly available for isolates at the website of NCBI. Data are illustrated annually from 2003 through November of 2013. Annual mean Replikin concentration (Replikin sequences per 100 amino acid residues) is illustrated with black columns. Standard deviation of the mean is illustrated in gray columns with caps. The data reflect cycles in Replikin concentration in csd gene in honeybees. Peaks are observable in 2008 and 2012. The peak in 2012 coincides with high levels of Colony Collapse Disorder in North America and Europe in 2013 and is incidental with 2011 peaks in Replikin concentration in Varroa destructor infestations in honeybees, Nosema infections in honeybees, other specific viruses in honeybees, and Replikin concentrations in ATPase in honeybees. The peak in 2008 is coincidental with Replikin concentrations in viruses in honeybees analyzed by applicants.

FIG. 15 illustrates percent of publicly available genomic sequences of isolates of complementary sex-determination (csd) gene in honeybees analyzed by applicants for given time periods having Replikin concentrations (Replikin sequences per 100 amino acid residues) of greater than 4.0. The graph reflects analysis of the genomic or proteomic information publicly available for isolates at the website of NCBI. Data are illustrated from 2003 through November 2013. The data reflect peaks in the percent of isolates having a Replikin concentration of greater than 4.0 in 2003-2005, 2011, and 2103. The peaks in 2011 and 2013 are coincident with high levels of Colony Collapse Disorder in North America and Europe and coincident with similar peaks in Varroa destructor, Nosema spp., and ATPase in honeybees.

### DETAILED DESCRIPTION OF THE INVENTION

### Definitions

A “Colony Collapse Disorder factor” or “CCD factor” or related terms means a parasite, pathogen, or honeybee amino acid sequence associated with and/or related to Colony Collapse disorder. A CCD factor includes, but is not limited to, Varroa destructor, Acarapis species, Nosema species, Deformed wing virus, Israeli acute paralysis virus, Kashmir bee virus, Sacbrood virus, or protein, polypeptide, or peptide sequences in honeybees (including, but not limited to, Apis mellifera, Apis cerana, Apis dorsata (giant honeybee), Euglossa hemichlora (orchid bee), Bombus impatiens (common eastern bumble bee), Bombus terrestris (buff-tailed bumblebee), Apis florea (little honeybee), Melipona compressipes, or Megachile rotundata (alfalfa leafcutting bee)) associated with CCD. A protein, polypeptide, or peptide sequence in honeybees associated with CCD includes, but is not limited to, ATPase of a honeybee or any Replikin sequence homologue or functional fragment in the ATPase of a honeybee or csd gene of a honeybee or any Replikin sequence homologue or functional fragment of csd gene or a honeybee.

A “functional fragment” of a Replikin sequence as described herein is a fragment, variant, analog, or chemical derivative of a Replikin sequence that retains at least a portion of the immunological cross reactivity with an antibody specific for the Replikin sequence. A fragment of the Replikin sequence refers to any subset of the molecule. Variant peptides of the sequence may be made by direct chemical synthesis, for example, using methods well known in the art. An analog of a Replikin sequence to a non-natural protein or polypeptide is substantially similar to either the Replikin sequence of the protein or a fragment thereof. Chemical derivatives of a Replikin sequence contain additional chemical moieties.

As used herein, the term “preferentially binds” or “specifically binds” and related terms referencing the interaction of a binding molecule such as, for example, an antibody, and the structure to which it binds (antigen) means that the binding molecule preferentially recognizes the structure to which it binds even when present among other molecules (such as in a mixture of molecules). Specific or preferential binding of a binding molecule to a binding structure or an immunogenic portion of a binding structure is specific and preferential when the binding molecule binds to the structure or portion thereof and does not bind with the same level of affinity to other structures. Binding affinity may be determined by one of ordinary skill in the art using, for example, BIACORE, enzyme-linked immunosorbent assays, or radioimmuno assays. A binding molecule may cross-react with related antigens and preferably does not cross-react with affinity to unrelated antigens. Binding between a binding molecule and the structure to which it binds may be mediated by covalent or non-covalent attachment, or both.

As used herein a “vaccine” is any substance, compound, composition, mixture, or other therapeutic substance that, when administered to a honeybee or animal via any method of administration known to the skilled artisan now or hereafter, including by oral administration, produces a blocking effect, an immune response, an antibody response, or a protective effect in the honeybee or other animal.

As used herein, a “Replikin sequence” is an amino acid sequence of 7 to 50 amino acid residues comprising (1) a first lysine residue located six to ten residues from a second lysine residue; (2) at least one histidine residue; and (3) at least 6% lysine residues, where the sequence is the shortest sequence comprising the first and second lysine residues of element (1) and the at least one histidine of element (3). A Replikin sequence may comprise more than two lysine residues and more than one histidine residue so long as at least two of the lysine residues and at least one histidine residue reflect the requirements of the definition of a Replikin sequence. The term “Replikin sequence” can also refer to a nucleic acid sequence encoding a Replikin peptide sequence.

As used herein, an “isolated” peptide may refer to a peptide that is, after purification, substantially free of cellular material or other contaminating proteins or peptides from the cell or tissue source from which the peptide is derived, or substantially free from chemical precursors or other chemicals when chemically synthesized by any method, or substantially free from contaminating peptides when synthesized by recombinant gene techniques. An isolated or chemically synthesized peptide may be synthesized by organic chemical methods. An isolated peptide may also be synthesized by biosynthetic methods. An isolated peptide may also refer to a peptide that is, after purification, substantially free of cellular material or other contaminating proteins or peptides from the cell or tissue source from which the peptide is derived, or substantially free from chemical precursors or other chemicals when chemically synthesized by any method, or substantially free from contaminating peptides when synthesized by recombinant gene techniques. A protein or peptide that has been isolated in silico from nucleic acid or amino acid sequences available through public or private databases or sequence collections or analysis may be synthesized by chemical means or isolated from biological materials. An isolated peptide may be synthesized by biosynthetic or organic chemical methods.

Proteins, protein fragments, polypeptides, or peptides in this specification may be chemically synthesized by any method known to one of skill in the art now and hereafter. For example, isolated proteins, protein fragments, polypeptides, or peptides may be synthesized by solid phase synthesis. The production of these materials by chemical synthesis avoids the inclusion of (or the need to remove by purification) materials that are byproducts of other production methods such as recombinant expression or isolation from biological material. Such byproducts may include, for example, avian proteins associated with vaccines produced using birds' eggs, bacterial proteins associated with recombinant production in bacteria, or proteins or contaminants associated with any recombinant activity such as with productions of proteins or other sequences in insect cells.

“Homologous” or “homology” or “sequence identity” as used in this specification indicate that an amino acid sequence or nucleic acid sequence exhibits substantial structural equivalence with another sequence, namely, any Replikin peptide sequence (including SEQ ID NO(s): 1-43) identified in an isolate of a CCD factor or any nucleotide sequence encoding a Replikin peptide sequence in an isolate of a CCD factor (a redundancy in a coding sequence may be considered identical to a sequence encoding the same amino acid). To determine the percent identity or percent homology of an identified sequence, a sequence is aligned for optimal comparison purposes with any one of possible basis sequences. For purposes of this paragraph, a basis sequence is a Replikin sequence identified in an isolate of a CCD factor. Where gaps are necessary to provide optimal alignment, gaps may be introduced in the identified sequence or in the basis sequence. When a position in the identified sequence is occupied by the same amino acid residue or same nucleotide as the corresponding position in the basis sequence, the molecules are considered identical at that position (as used herein amino acid or nucleic acid “identity” is equivalent to amino acid or nucleic acid “homology”). To determine percent homology, the amino acid residues or nucleotides at corresponding amino acid positions or nucleotide positions are compared between the identified sequence and the basis sequence. The total number of amino acid residues or nucleotides in the identified sequence that are identical with amino acid residues or nucleotides in the basis sequence is divided by the total number of residues or nucleotides in the basis sequence (if the number of residues or nucleotides in the basis sequence is greater than the total number of residues or nucleotides in the identified sequence) or by the total number of amino acid residues or nucleotides in the identified sequence (if the number of residues or nucleotides in the identified sequence is greater than the total number of residues or nucleotides in the basis sequence). The final number is determined as a percentage. As such, the percent identity between the two sequences is a function of the number of identical positions shared by the sequences, taking into account the number of gaps (where a gap must be introduced for optimal alignment of the two sequences) and the length of each gap. Any structural or functional differences between sequences having sequence identity or homology will not affect the ability of the sequence to function as indicated in the desired application.

For example, SEQ ID NO: 21 (KKRDDHLK) is considered more than 87% homologous with SEQ ID NO: 22 (KRDDHLK). The more than 87% homology between SEQ ID NO(s): 21 and 22 is determined as follows: SEQ ID NO: 21 is the basis sequence. Upon alignment, SEQ ID NO: 21 is identical to SEQ ID NO: 22 in all positions except the N-terminal lysine of SEQ ID NO: 21. To determine percent homology, the seven aligned identical residues are divided by the total number of residues in SEQ ID NO: 21, namely eight residues, giving 0.875 or more than 87% homology. SEQ ID NO: 22 is likewise a functional fragment of SEQ ID NO: 21.

SEQ ID NO: 21 (KKRDDHLK) is more than 44% homologous with SEQ ID NO: 24 (KIDRWFLHK) in that the lysine residues in position 1 at the N-terminus are identical, the aspartic acid residues in position 3 from the N-terminus are identical, the leucine residue at position six from the N-terminus of SEQ ID NO: 21 is identical to the leucine residue at position seven of SEQ ID NO: 24 (the phenylalanine residue at position 6 in SEQ ID NO: 24 is considered a gap), and the lysine residue at the C-terminus of both sequences are identical (the histidine residue at position 8 of SEQ ID NO: 24 is considered a gap). SEQ ID NO: 24 is the basis sequence. Four residues are identical between SEQ ID NO(s): 21 and 24 over nine total residues in SEQ ID NO: 24 giving 0.444 or more than 44% homology.

SEQ ID NO: 3 (KQNLKLLETKH) is more than 63% homologous with SEQ ID NO: 4 (KLLETKHITEK) in that positions 1-7 of SEQ ID NO: 4 are identical with positions 5-11 of SEQ ID NO: 3. Both SEQ ID NO(s): 3 and 4 have eleven total residues. With seven identical positions over eleven total residues, the sequences are 0.6363 or more than 63% homologous.

SEQ ID NO: 25 (KLSHDVLLRAK) is more than 90% homologous with SEQ ID NO: 27 (KLSHDVLLHAK) in that the only residues that do not match are at position nine in both sequences (namely, an arginine residue and a histidine residue). The sequences are eleven residues long. With ten identical residues over eleven total residues, the sequences are 0.90909 or more than 90% homologous.

To determine homology between an identified sequence that is contained in a larger polypeptide, protein fragment, or protein, and a basis sequence, the polypeptide, protein fragment, or protein must first be optimally aligned with the basis sequence. Upon alignment of the sequences, the residue in the identified sequence that is farthest to the amino-terminus of the polypeptide, protein fragment, or protein and identical to a residue in the basis sequence that is farthest to the amino-terminus of the basis sequence is considered the amino-terminal residue of the identified sequence. Likewise, upon alignment, the residue in the identified sequence that is farthest to the carboxy-terminus of the polypeptide, protein fragment, or protein and identical to a residue in the basis sequence that is farthest to the carboxy-terminus of the basis sequence is considered the carboxy-terminal residue of the identified sequence.

Concerning gaps, the number of gaps in either the basis sequence or the identified sequence should be limited to the number of gaps allowable without significantly compromising the function of the identified sequence as compared to the basis sequence. In general, many gaps in the sequence of the basis peptide or in the sequence of the identified peptide are allowed based on homology as defined herein. Relatively more gaps are allowed if the lysines and histidines that create the definition of the Replikin peptide are identically shared between the basis peptide and the identified peptide. Relatively more gaps are also allowed if the lysines and histidines that create the definition of the Replikin peptide are shared at least in close position (for example within ten, nine, eight, seven, six, five, four, three, two, or one amino acid residue). If some of the lysine residues and histidine residues that create the definition of the Replikin peptide are not present in the identified peptide, fewer gaps may be allowed. Nevertheless, if the identified peptide functions similarly to the basis peptide, any number of gaps is allowed. In general, three or more gaps are allowed in the sequence of the basis peptide or in the sequence of the identified peptide within ten amino acid residues of the basis peptide if no lysines or histidines are present in the identified peptide. Two or more gaps or one or more gaps are also allowed. Nevertheless, if the identified sequence provides the same or a similar function to the basis sequence, more gaps are allowed up to the number of gaps that will provide a homology of 30%, 40%, 50%, 60%, 70%, 80%, 90%, 95%, or more homology. Additionally, where the lysines and histidines of the Replikin definition are present in both the identified peptide and the basis peptide, there should be no limit on how many gaps are allowed.

The presence of lysines and histidines providing for the Replikin definition in an identified peptide requires significantly less homology because the lysines and the histidines of the Replikin definition provide for conservation of Replikin function. For example, in Table 8 and the description thereof in columns 62 and 63 in U.S. Pat. No. 7,442,761, a highly mutable tat protein in HIV is described and analyzed. As may be seen from Table 8 in U.S. Pat. No. 7,442,761, in tat protein of HIV, which is essential for replication in the virus, lysines and histidines that are essential to maintaining the Replikin definition within a key Replikin peptide in the protein are observed to be 100% conserved, while substitutions in amino acid residues that are not essential to maintaining the Replikin definition are commonly substituted. The conservation of the key amino acids for maintaining the Replikin definition is understood to provide a specific survival function for HIV. The same phenomenon is seen in influenza. See U.S. Pat. No. 7,442,761, column 62, lines 42-45.

As used herein, “Replikin Count” or “Replikin concentration” refers to the number of Replikin sequences per 100 amino acids in a protein, protein fragment, virus, or organism. A higher Replikin concentration in a first strain of a virus or organism has been found to correlate with more rapid replication of the first virus or organism as compared to a second, earlier-arising or later-arising strain of the virus or organism having a lower Replikin concentration. Replikin concentration is determined by counting the number of Replikin sequences in a given sequence, wherein a Replikin sequence is a peptide of 7 to 50 amino acid residues comprising (1) a first lysine residue six to ten residues from a second lysine residue, (2) at least one histidine residue, (3) and 6% or more lysine residues where the Replikin sequence is the shortest sequence comprising the first and second lysine residues of element (1) and the at least one histidine residue of element (2). A Replikin sequence may comprise more than two lysine residues and more than one histidine residue so long as there is at least one lysine residue six to ten residues from a second lysine residue and at least one histidine residue. Counting Replikins sequences within a polypeptide to determine Replikin concentration includes counting all sequences that fit the definition of a Replikin sequence, including counting all adjacent and overlapping Replikin sequences, and includes counting all sequences within the identified polypeptide, dividing by the number of amino acid residues present in the polypeptide, and multiplying by 100 to arrive at the Replikin concentration, or number of Replikin sequences per 100 amino acid residues. A Replikin sequence for the purpose of determining Replikin concentration as described in this paragraph may also be a nucleic acid that encodes a Replikin peptide sequence defined according to this paragraph.

### Synchronous Increase in Replikin Concentration in Factors Causing Colony Collapse Disorder in Honeybees

In review of various factors associated with Colony Collapse Disorder in honeybees (CCD), Applicants have surprisingly discovered that Replikin concentration is coordinated in multiple species related to CCD. Coordination across various factors associated with CCD is, until now, both unexpected and unusual in Replikin-sequence function. Applicants observed that the Replikin class of genomic structures increases together in synchrony in several previously unrelated species but for their association and involvement in CCD. Previously unknown, the present demonstration of synchronous waxing and waning in Replikin gene structures of each organism, whether a known mite carrier of viruses in CCD, a fungus, a bacteria, or other parasites or pathogens, provides specific Replikin structures that may be targeted with solid-phase synthesis for production of safe effective vaccines and replication blockers.

### Two Cycles in Replikin Concentration in Factors Causing Colony Collapse Disorder in Honeybees

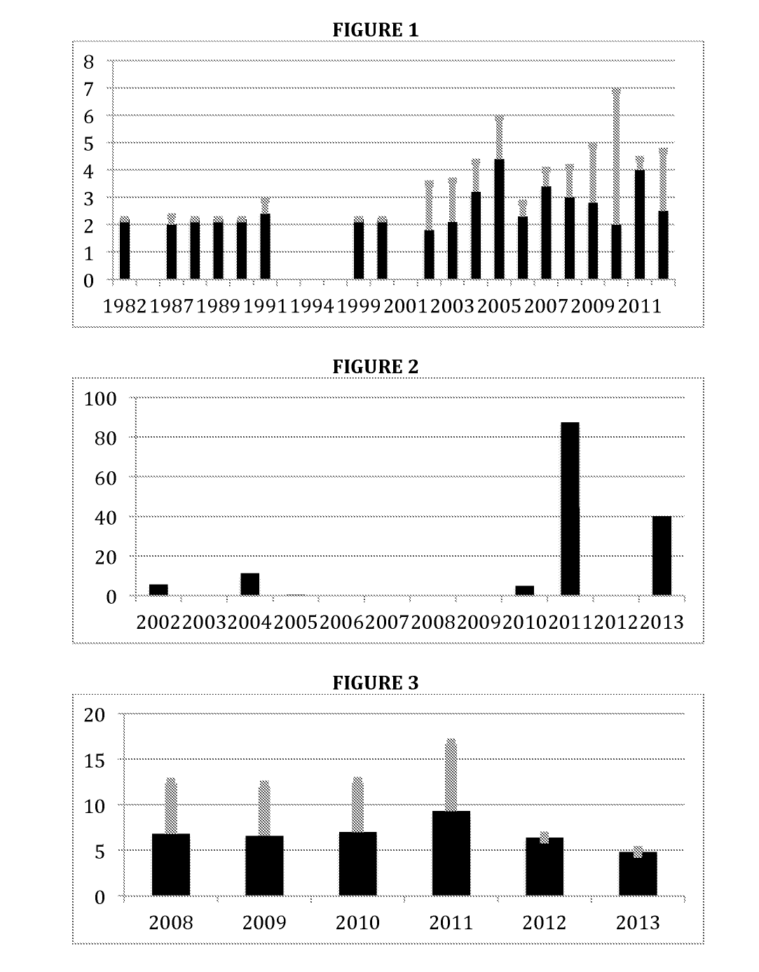

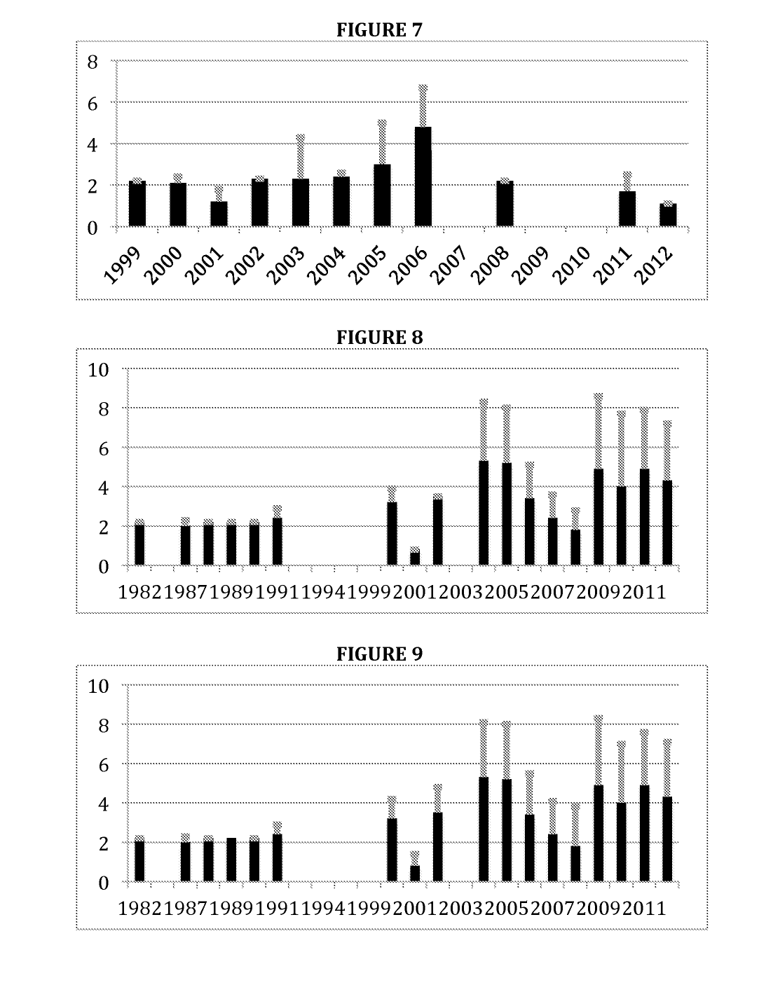

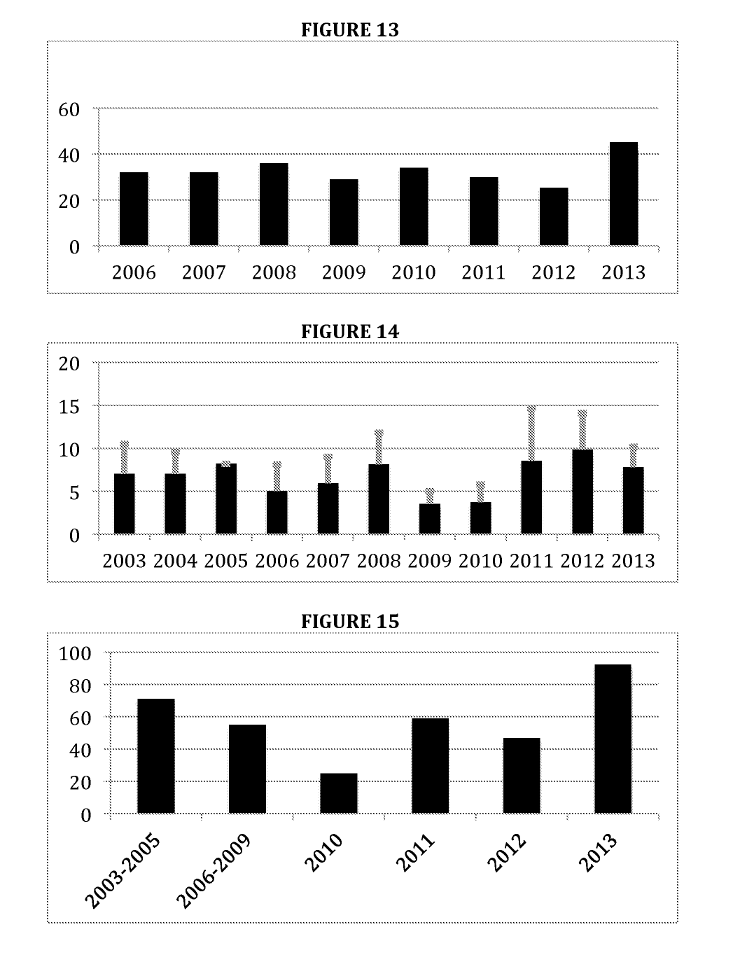

In review of various factors associated with CCD, Applicants have surprisingly discovered that Replikin concentration is coordinated in multiple species related to CCD. These species were observed to carry the same message, the Lethal Replikins Gene (LRG). Among the various species associated with CCD, Replikin concentrations were observed to increase markedly from 2007 through 2008 and again from 2012 to 2013. See, e.g., FIGS. 1-12. These years were associated with large losses from CCD. See FIG. 13. During less active honeybee colony loss, the standard deviation from the mean Replikin concentration in all three factors (Varroa destructor, Nosema spp., and honeybee viruses) was observed to be small or absent while during years in which large losses of honeybee colonies were observed, the standard deviation of the mean increased markedly prior to an increase in Replikin concentration and a concomitant increase in honeybee colony loss.

Both the Replikin concentration and the timing of the cycles of Replikin concentration are coincident in Varroa destructor, Nosema species, in the observed bee viruses, and in ATPase and csd gene of bees themselves. This timing of cycles among the species associated with CCD points to the participation of each in the disorder and, more importantly, points to the role of the Lethal Replikin Gene among the species in the disorder. This knowledge allows for targeting Replikin sequences identified as associated with the disorder in these various species and allows for the vaccines designed herein against the disorder.

Low Percentages of Apis dorsata (Black Honeybee) have Replikin Sequences in Reported Amino Acid Sequences

The black honeybee (Apis dorsata) is reported to have resistance to CCD. Applicants reviewed amino acid sequences disclosed at National Center for Biotechnology Information for black honeybees. Only 36% of black honeybee isolates had at least one Replikin sequence in the reported amino acid sequence. Those isolates where a Replikin sequence was observed were generally Himalayan black honeybees and these bees were found to have very high Replikin concentrations. German black honeybees, on the other hand, were found to have very low Replikin concentrations.

Replikin sequences from black honeybees, functional fragments, and homologues are useful as part of formulations of vaccines against CCD. Replikin sequences from Himalayan black honeybees, functional fragments, and homologues are particularly useful in formulations of vaccines. Targeting of Replikin sequences, fragments, and homologues provides for control of CCD.

A large outbreak of CCD was reported in Himalayan black honeybees in 2012. 100% of isolates from Himalayan black honeybees in 2012 were observed to have Replikin concentrations over 4.0

Shared and Conserved Replikin Peptide Sequences and their Homologues

Replikin sequences and their homologues provided by an aspect of the invention may be identified in any CCD factor including any strain of a CCD factor known now or identified or known hereafter. Compounds of the invention may be conserved within a CCD factor, across types of CCD factor, within strains of a CCD factor, and across strains of a CCD factor. The compounds, because they are Replikin sequences, related to Replikin sequences, derived from Replikin sequences, identified as comprising Replikin sequences, or designed to comprise Replikin sequences, are related to rapid replication, virulence, and lethality in a CCD factor and comprise necessary structure for replication blocking and antigenicity. These characteristics of Replikin sequences have been previously established in other viruses and organisms (see, e.g., U.S. Pat. No. 7,894,999, U.S. Pat. No. 7,758,863 and WO 2008/143717) but have not previously been disclosed in a CCD factor and the surprisingly effective utility of the Replikin sequence in predictions and therapies in a CCD factor is established herein. Compounds of the invention, including conserved Replikin peptides, are useful as blocking compounds to block rapid replication of CCD factors and as immunogenic compounds to stimulate the immune system of a subject to produce an immune response, which may include production of antibodies or other binding molecules. These compounds are useful as blocking compounds and to stimulate blocking mechanisms. Compounds of the invention are also useful in therapies such as vaccines. Compounds of the invention are likewise useful in producing antibodies, antibody fragments, or other binding or antagonizing agents, which may be used, among other things, for diagnostic and therapeutic purposes, including passive immunity.

The immunogenic compounds, antibodies (and other binding or antagonizing agents), blocking agents, and vaccines of the invention are useful against CCD including against any CCD factor. The compounds of the invention are also useful for diagnostic purposes, including identifying rapidly replicating, virulent, or lethal strains of virus.

Information on the conservation of homologous sequences across various CCD factors and in different regions provides sequences that offer immunogenic and blocking compounds for antagonism of all strains comprising these homologues and across all regions having strains comprising these homologues. As a result, a vaccine is provided herein that offers cross-strain protection for a variety of strains of a CCD factor.

Replikin peptides in general are seen to be conserved across CCD factors. The key amino acid residues that provide for the Replikin sequence structure are the lysine and histidine residues wherein a Replikin sequence has at least one lysine on one terminus and at least one lysine or one histidine on the other terminus, at least one lysine that is six to ten residues from at least one other lysine, at least one histidine, and at least six percent lysines in total between the terminal lysine and the terminal lysine or histidine.

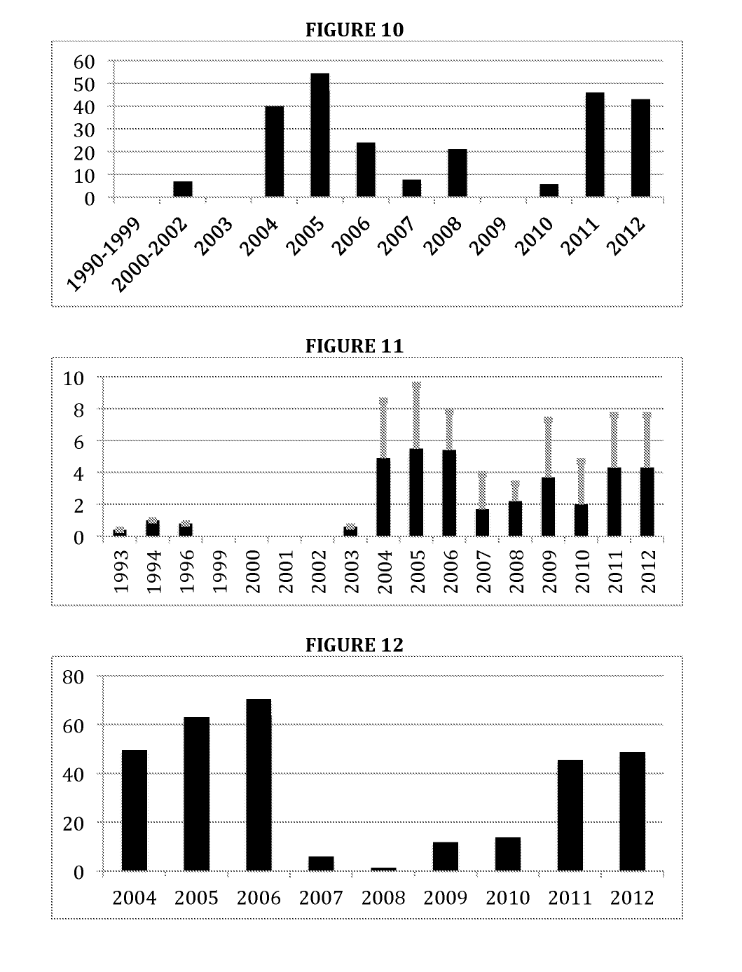

As may be seen in FIG. 10 of WO 2005/104754, when conserved homologous Replikin sequences are aligned one on top of the other over time, it is most apparent that fixed and conserved portions of the structure of Replikin sequences align in a series of posts or girders that illustrate, like the structure of a building, how key conserved amino acids provide constancy for the survival of a virus such as a CCD factor over time as it mutates to avoid immune recognition in its prospective host but maintains key functional genetic structures that provide for continued replication of the virus. These key functional genetic structures provide targets antagonized by Replikin-based therapies.

### Compounds and Compositions Comprising Peptides Homologous to CCD Factor Replikin Peptides

One aspect of the present invention provides a protein, a protein fragment, a polypeptide, or a peptide that comprises at least one peptide A homologous with at least one Replikin peptide identified in an isolate of a CCD factor. The Replikin peptide may be any Replikin peptide identified in an isolate of a CCD factor. The Replikin peptide may further be a Replikin peptide identified as conserved across strains or across regions in isolates of a CCD factor or any homologue of a Replikin peptide identified as conserved across strains or across regions in isolates of a CCD factor. For example, the Replikin peptide may be any one of SEQ ID NO(s): 1-43 or any homologue of any one of SEQ ID NO(s): 1-43 or any functional fragment of a Replikin sequence, such as, for example, SEQ ID NO(s): 1-43.

Peptide A of the protein, protein fragment, polypeptide, or peptide may be 30%, 40%, 44%, 50%, 60%, 63%, 70%, 80%, 87%, 90%, or 95% or more homologous or 100% homologous with a Replikin peptide, including any of the peptides of SEQ ID NO(s): 1-43. A protein fragment or peptide may likewise be a peptide that consists of a peptide A that is homologous with a Replikin peptide of a CCD factor, including any of SEQ ID NO(s): 1-43. A peptide consisting essentially of or consisting of a Replikin peptide of a CCD factor, including any one of SEQ ID NO(s): 1-43, is also provided.

The amino acid sequence of the provided isolated or synthesized protein, protein fragment, polypeptide, or peptide may partially match an amino acid sequence of an expressed whole protein. At least one, five, ten, twenty, thirty, forty, fifty, one hundred, two hundred, three hundred, four hundred, five hundred, five hundred and fifty or more amino acid residues of the amino acid sequence of the expressed whole protein may not be present in the protein, protein fragment, polypeptide, or peptide. The amino acid sequence of an isolated or synthesized protein fragment, polypeptide, or peptide may also partially match the amino acid sequence of an expressed whole protein where at least one, ten, twenty, thirty, forty, fifty, sixty, seventy, eighty, ninety, one hundred, one hundred fifty, two hundred, two hundred fifty, three hundred, three hundred fifty, four hundred, four hundred fifty, five hundred, five hundred fifty or more amino acid residues of at least one terminus of the amino acid sequence of the expressed whole protein is (are) not present. Any additional number of amino acids may be situated on one or the other terminus or on both termini of the protein fragment, polypeptide, or peptide. An isolated or chemically-synthesized peptide comprising a Replikin sequence, homologues of a Replikin sequence, or functional fragment of a Replikin sequence may be 10, 20, 30, 40, 50, 60, 70, 80, 90, or 100 or more residues in total length.

Because a Replikin peptide, such as SEQ ID NO(s): 1-43, is associated with rapid replication, infectivity, and/or lethality in functional proteins in a CCD factor and because a Replikin peptide such as any one of SEQ ID NO(s): 1-43 are blocking agents and/or antigenic, inclusion of any Replikin peptide, homologue, or function fragment thereof, in a protein, protein fragment, polypeptide, or peptide does not negate the functional nature of the Replikin peptide. As such, antagonism of at least one Replikin peptide, including at least one of SEQ ID NO(s): 1-43, functional fragment of SEQ ID NO(s): 1-43, or a homologue of SEQ ID NO(s): 1-43 (with homology of 30% or greater) within a protein, protein fragment, polypeptide, or peptide would be expected to antagonize the replication, infectivity, and/or lethality of the protein fragment, polypeptide, or peptide.

A provided peptide may further be a peptide B of 7 to about 50 amino acid residues where peptide B contains a peptide A that is 30%, 40%, 50%, 60%, 70%, 80%, 90%, or 95% or more homologous or 100% homologous with any Replikin peptide, including one of SEQ ID NO(s): 1-43. A non-limiting peptide may further be a peptide A that is a Replikin peptide.

One aspect of the invention provides a biosynthetic composition comprising a Replikin peptide, homologue, or functional fragment. In a non-limiting embodiment, the isolated protein fragment, polypeptide, or peptide of an aspect of the invention is chemically synthesized by solid phase methods.

An isolated or synthesized polypeptide or peptide may comprise a peptide A that has about the same number of amino acid residues as a peptide B, where peptide B is one of the peptides of SEQ ID NO(s): 1-43 and where the lysine residues and histidine residues in peptide A are conserved as compared to the lysine residues and histidine residues in peptide B. Peptide A may have additional amino acid residues and may have a length of up to 100 residues. Some of the additional amino acid residues may be positioned within the lysine or histidine termini of peptide A so long as a level of homology is maintained between peptide A and peptide B that retains at least some of the functionality of the Replikin peptide of peptide B. Functionality may include, but is not limited to, antigenicity, rate of replication, antagonizability of a protein containing said peptide A or said peptide B, binding capacity of binding agents to peptides A or B, etc.

An isolated or synthesized polypeptide or peptide may also comprise up to about 90, about 80, about 70, about 60, about 50, about 40, about 30, about 20, about 10, about 5, about 4, about 3, about 2, or about 1 additional amino acid residues. The residues may be entirely outside of the Replikin structure or entirely within the Replikin structure or partially within and partially outside the Replikin structure.

All of the above-discussed proteins, protein fragments, polypeptides, and peptides comprise the functional unit of a homologue of a Replikin peptide present in or isolated from a CCD factor. The Replikin peptide may be any one of SEQ ID NO(s): 1-43. Antagonism of any of the homologues of a Replikin peptide will antagonize replication in a CCD factor. As a result, the proteins, protein fragments, polypeptides, and peptides are useful as blocking compounds or immunogenic compounds, therapeutic compounds, vaccines, and for other therapies directed to antagonizing the replication and/or lethality of a strain of a CCD factor. When comprised in a vaccine, disclosed proteins, protein fragments, polypeptides, and peptides are expected to be capable of limiting the excretion or shedding of a CCD factor such that the virus, parasite, or fungus is limited in its spread from host to host or from host to reservoir to host, etc. As such, disclosed compounds are effective at limiting sources of a CCD factor infection. Likewise, any binding agent that binds one of the proteins, protein fragments, polypeptides, and peptides discussed above will antagonize the replication and/or lethality of a strain a CCD factor and limit sources of a CCD factor infection such as transmission from host to host or from host to reservoir to host.

### Compositions Targeting Peptide Sequences, Functional Fragments, and Homologues in CCD Factors to Control CCD

A protein, protein fragment, or peptide comprising at least one Replikin peptide sequence present or identified in an isolate of a CCD factor is an excellent component in a compound for targeting rapid replication of viruses, parasites, fungi, or bacteria that are factors in colony collapse. The protein, protein fragment, or peptide may be comprised in an immunogenic or blocking compound. The at least one Replikin sequence (fragment or homologue thereof) of the protein, protein fragment, or peptide provides a blocking mechanism for controlling rapid replication of factors involved in CCD. The Replikin sequence (fragment or homologue thereof) likewise provides an immunogenic target against which the immune system of a subject responds to control rapid replication of factors of CCD and CCD itself. Because at least a functional portion of the structure of the target is maintained in a functional fragment of the at least one Replikin sequence, a functional fragment of the Replikin sequence is a good target against which a blocking or immune system of a subject responds and through which replication of a pathogen, parasite, fungus, bacteria, etc., may be blocked. The compound may comprise a plurality of synthesized or isolated Replikin sequences.

### Vaccines Comprising Peptides Homologous to CCD Factor Replikin Peptides

A blocking or immunogenic compound provided as an aspect of the invention may be used as a component of a non-limiting vaccine against any strain of a CCD factor and/or against CCD itself. A vaccine comprising one or more homologues of a Replikin peptide of a CCD factor may be used against any CCD factor or against CCD. The vaccine may comprise one or more homologues of SEQ ID NO(s): 1-43. Likewise, a vaccine comprising one or more homologues of a Replikin peptide may be used against a CCD factor and may antagonize the replication and/or lethality of a CCD factor. Further, mixtures of homologues of SEQ ID NO(s): 1-43 are provided as vaccines to antagonize CCD and the replication and/or lethality of a CCD factor. Such vaccines are useful for antagonizing replication, lethality, and excretion or spread of a CCD factor.

In another non-limiting embodiment, the vaccine comprises a mixture of peptides, such as a the mixture comprising isolated or synthesized peptides of SEQ ID NO(s): 1-43. A vaccine may comprise an approximately equal molar mixture of the isolated or synthesized peptides of SEQ ID NO(s): 1-43. In a further non-limiting embodiment, the vaccine comprises approximately equal weight of the isolated or synthesized peptides of SEQ ID NO(s): 1-43.

A vaccine may comprise a plurality of the shortest Replikin peptides from any region of the genome of a CCD factor including the VP1, VP2, or VP3 gene area. A vaccine may comprise the shortest Replikin peptides from a gene area identified in a CCD factor isolate or a plurality of a CCD factor isolates predicted to have a greater lethality than at least one other isolate of a CCD factor. A vaccine may further comprise a plurality of the longest Replikin peptides from any gen area of the virus including the VP1, VP2, or VP3 gene area identified in a CCD factor isolate or a plurality of a CCD factor isolates. A vaccine may also comprise a mixture of the shortest and longest Replikin peptides.

A vaccine may be formulated with a pharmaceutically acceptable excipient, carrier, or adjuvant. One pharmaceutically acceptable carrier or excipient is water. Excipients, carriers, or adjuvants may include, but are not limited to, excipients, carriers and adjuvants known to those of skill in the art now or hereafter.

A composition of the invention may be formulated for delivery by any available route including, but not limited oral, nasal, bronchial, transdermal, transmucosal, or any other routes. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions. Administration of the vaccine via any method may produce a blocking response in a honeybee or other animal, it may further produce an immune response including, for example, an antibody or antibody-like response. In a further non-limiting embodiment, the vaccine may produce a protective effect.

Generally, the dosage of peptides is in the range of from about 0.01 μg to about 500 mg, from about 0.05 μg to about 200 mg, about 0.075 μg to about 30 mg, about 0.09 μg to about 20 mg, about 0.1 μg to about 10 mg, about 10 μg to about 1 mg, and about 50 μg to about 500 μg. The skilled practitioner can readily determine the dosage and number of dosages needed to produce an effective blocking response, immune response, or protective effect.

Advance information concerning Replikin peptides in expanding colony collapses allows for the rapid production of specific effective synthetic vaccines using one, or a combination, of Replikin peptides. Such synthetic vaccines have been demonstrated in rabbits, chickens, and shrimp. See, e.g., Examples 6 and 7 of U.S. application Ser. No. 11/355,120, filed Feb. 16, 2006, and Example 2 of U.S. application Ser. No. 12/108,458, filed Apr. 23, 2008, see also Examples 2, 3, 4, etc., herein.

### Antibodies as Diagnostics and Therapies for Identified Replikin Sequences

In another aspect of the invention, isolated Replikin peptides may be used to generate antibodies, antibody fragments, or to generate or identify other binding agents, which may be used, for example, for diagnostic purposes or to provide passive immunity in an individual. See, e.g., U.S. Pat. No. 7,894,999 and WO 2008/143717 (each incorporated herein by reference in their entirety). Various procedures known in the art may be used for the production of antibodies or antibody-like proteins to Replikin sequences, homologues, or functional fragments thereof, or to proteins, protein fragments, polypeptides, or peptides comprising Replikin sequences, homologues, or functional fragments thereof. Such antibodies include, but are not limited to, polyclonal, monoclonal, chimeric, humanized, single chain, Fab fragments and fragments produced by a Fab expression library. Antibodies that are linked to a cytotoxic agent or signaling moiety may also be generated. Antibodies may also be administered in combination with an antiviral agent. Furthermore, combinations of antibodies to different Replikins may be administered as an antibody cocktail.

Binding agents are provided including an antibody, antibody fragment, or binding agent that binds to at least at least one Replikin peptide or homologue of a Replikin sequence or functional fragment of a Replikin sequence of a CCD factor, which may include, for example, at least one Replikin peptide of SEQ ID NO(s): 1-43.

### Nucleic Acids and Compositions of Nucleic Acids

An isolated or synthesized nucleic acid sequence is also provided that encodes a at least one Replikin peptide of a CCD factor. The at least one Replikin peptide may be, and is not limited to, any peptide of SEQ ID NO(s): 1-43. An aspect further provides an immunogenic composition that comprises an isolated or synthesized nucleic acid provided above. An aspect further provides a vaccine against a CCD factor comprising an isolated or synthesized nucleic acid provided above.

### Methods of Predicting Increases in CCD

One non-limiting aspect of the present invention provides a method of predicting increases in CCD comprising, respectively, identifying an increase in the percentage of isolates of a CCD factor or plurality of CCD factors having a Replikin concentration (number of Replikin sequences per 100 amino acid residues) greater than 4.0 between two time periods or identifying a decrease in the percentage of isolates of one or more CCD factors having a Replikin concentration (number of Replikin sequences per 100 amino acid residues) greater than 4.0 between two time periods. In a non-limiting embodiment, more than two time periods are compared and the percentage of isolates in the more than two time periods shows a pattern of increase or decrease.

In a further non-limiting embodiment, a plurality of isolates in a given time period may be analyzed for Replikin concentration. The percentage of isolates having a Replikin concentration of greater than 4.0 Replikin sequences per 100 amino acid residues in a first time period may be compared to the percentage of isolates having a Replikin concentration of greater than 4.0 Replikin sequences per 100 amino acid residue in a second time period. If the later timer period has a higher percentage of isolates having a Replikin concentration of greater than 4.0 then it is predicted that CCD incidence will expand. If the later time period has a lower percentage of isolates having a Replikin concentration of greater than 4.0 then it is predicted that the CCD incidence will retract and an outbreak is expected to decrease in severity.

For example, FIG. 10 provides data demonstrating a marked increase in percent of isolates of viruses of honeybees associated with CCD having a Replikin concentration of greater than 4.0 around 2005 (predicting an increase) and a marked increase in percent of isolates having a Replikin concentration of greater than 4.0 between around 2012 (predicting an increase).

One non-limiting aspect of the present invention provides a method of determining an increased probability of increased CCD incidence within about six months to about three years following an increase in Replikin concentration in an isolate of a CCD factor or multiple CCD factors comprising identifying an increase in the concentration of Replikin sequences in at least one first isolate of a CCD factor as compared to at least one other isolate of a CCD factor wherein said at least one first isolate is isolated later than said at least one other isolate is isolated, and wherein said increase in the concentration of Replikin sequences signifies the increased probability of increased incidence of CCD within about six months to about three years following the increase in the concentration of Replikin sequences. In a non-limiting embodiment, the first isolate of a CCD factor is isolated at least about six months later than the at least one other isolate.

In a further non-limiting embodiment of the aspect of the invention, the method of prediction further comprises comparison of the standard deviation of the mean of Replikin concentrations of isolates of a CCD factor or plurality of CCD factors from a given time period, such as a given month, a given year, or any other given time period. In a further non-limiting embodiment the Replikin concentration is a mean Replikin concentration of a plurality of isolates with standard deviation from the mean and the standard deviation from the mean is greater than the standard deviation from the mean Replikin concentration of a plurality of other isolates.

For example, FIG. 1 demonstrates a marked increase in both the mean and the standard deviation of the mean for Replikin concentration in 2005 in Varroa destructor. Both increased Replikin concentration and increased standard deviation from the mean Replikin concentration in 2005 correlate with increased incidence of CCD around 2007 to 2008. This correlation has been seen in other pathogens including, for example, influenza, malaria, taura syndrome virus, white spot syndrome virus, foot and mouth disease, and other diseases. See, e.g., FIGS. 1-21 in WO 2008/143717.

### Computer Methods for Predicting Incidence of CCD

A prediction of expansion or retraction of CCD incidence population may be performed by a processor. A prediction may be output to a user or display. Likewise, a particular Replikin peptide or Replikin Peak Gene within an isolate or population of isolates of one or more CCD factors predicted to be expanding or retracting in replication or lethality may be identified and output to a user or display. A machine-readable storage medium may contain executable instructions that, when executed by a processor, cause the processor to provide sufficient data to a user, a printout, or a display such that the user or a user of the printout or display may predict increase or decrease in incidence of CCD. A non-limiting computer readable medium may be non-transitory. Software comprising methods of the invention and related data may be carried on a signal.

A computer system may include a processor coupled to a network, and a memory coupled to a processor, wherein the memory contains a plurality of instruction to perform the methods of prediction discussed herein. A user of outputted data from a processor, storage medium, machine-readable medium, or computer system may include any person or any machine that records or analyzes the outputted data. A display or printout may include any mechanism by which data is outputted so that any person or any machine may record or analyze the outputted data, including a printed document, a visual impulse, an aural impulse, or any other perceivable impulse, a computer monitor, a set of numbers, or any other display or printout of data including a digital recording medium.

### Example 1

### Two Cycles in Replikin Concentration in Factors Causing Colony Collapse Disorder in Honeybees

Applicants reviewed amino acid sequences disclosed at the website of NCBI from isolates of various factors associated with Colony Collapse Disorder (CCD) in honeybees for Replikin sequences and for concentrations of Replikin sequences in the disclosed sequences. Applicants reviewed amino sequences from Varroa destructor, Nosema spp., Deformed wing virus, Israeli acute paralysis virus, Kashmir bee virus, and Sacbrood virus. Applicants also reviewed amino acid sequences from ATPase of honeybees (Apis cerana and Apis mellifera). Applicants analyzed the sequences for mean Replikin concentration and standard deviation from the mean for years in which amino acid sequences were available.

Surprisingly, Applicants discovered that Replikin concentration is coordinated in multiple species related to CCD. These species were observed to carry the same message, the Lethal Replikins Gene (LRG). Among the various species associated with CCD, Replikin concentrations were observed to increase markedly from 2007 through 2008 and again from 2012 to 2013. See, e.g., FIGS. 1-12. These years were associated with large losses from CCD. See FIG. 13. During less active honeybee colony loss, the standard deviation from the mean Replikin concentration in all three factors (Varroa destructor, Nosema spp., and honeybee viruses), the standard deviation of the mean was observed to be small or absent while during years in which large losses of honeybee colonies were observed, the standard deviation of the mean increased markedly prior to an increase in Replikin concentration and a concomitant increase in honeybee colony loss.

Both the Replikin concentration and the timing of the cycles of Replikin concentration are coincident in Varroa destructor, Nosema species, in the observed bee viruses, and in ATPase of bees themselves. This timing of cycles among the species associated with CCD points to the participation of each in the disorder and, more importantly, points to the role of the Lethal Replikin Gene among the species in the disorder. This knowledge allows for targeting Replikin sequences identified as associated with the disorder in these various species and allows for the vaccines designed herein against the disorder.

Applicants identified the annual mean Replikin concentration, standard deviation of the mean, and percentage of isolates having a Replikin concentration above 4.0 Replikin sequences per 100 amino acid residues for Varroa destructor mite isolates with amino acid sequence data available at PubMed. The data are illustrated in FIGS. 1 and 2.

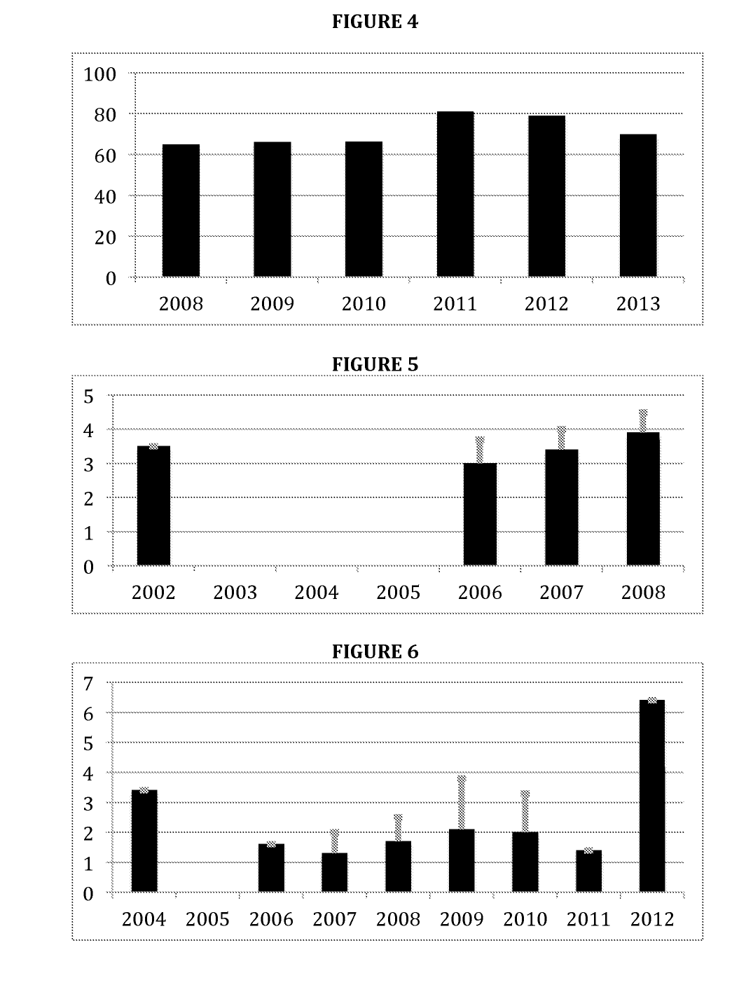

Applicants identified the annual mean Replikin concentration, standard deviation of the mean, and percentage of isolates having a Replikin concentration above 4.0 Replikin sequences per 100 amino acid residues for Nosema fungus isolates with amino acid sequence data available at PubMed. The data are illustrated in FIGS. 3 and 4.

Applicants identified annual mean Replikin concentration and standard deviation of the mean for virus isolates associated with CCD with amino acid sequence data available at PubMed analyzed by applicants. The data are illustrated in FIGS. 5-8.

Applicants identified annual mean Replikin concentration, standard deviation of the mean, and percentage of isolates having a Replikin concentration above 4.0 Replikin sequences per 100 amino acid residues for virus isolates associated with CCD with amino acid sequence data available at PubMed analyzed by applicants. The data are illustrated in FIGS. 9 and 10. Years without data reflect identification of no isolates.

Applicants identified annual mean Replikin concentration, standard deviation of the mean, and percentage of isolates having a Replikin concentration above 4.0 Replikin sequences per 100 amino acid residues for isolates of bee ATPase with amino acid sequence data available at PubMed analyzed by applicants. The data are illustrated in FIGS. 11 and 12. Years having no data reflect years in which no isolates were identified.

FIG. 13 illustrates data on losses of colonies of honeybees in the United States from 2066 through 2013.

### Example 2

### Synthetic Replikin Vaccine Against BCC

A synthetic vaccine was designed against BCC comprising solid-phase synthesized peptides of SEQ ID NO(s): 1-43 in approximately equal parts by weight. The peptides are combined in sterile water and sugar is added to the mixture. The vaccine is directed against factors causing Colony Collapse Disorder (BCC) including Varroa destructor, Nosema spp., deformed wing virus, Israeli acute paralysis virus, Kashmir bee virus, and Sacbrood virus. The vaccine is presented to a plurality of colonies of bees for consumption. The colonies are monitored for CCD.

### Example 3

### Replikin Sequences Conserved in Varroa destructor

Applicants reviewed the website of the NCBI for amino acid sequences isolated from Varroa destructor comprising Replikin sequences. Each Replikin sequence was identified. Applicants then compared Replikin sequences identified in amino acid sequences from Varroa destructor for conservation in isolates across time. The following sequences were identified as conserved. As disclosed below, the year of publication of the accession number of each amino acid sequence, the accession number, and the position of the Replikin sequence within the amino acid sequence at PubMed is identified. The listing reflects all occurrences of each Replikin sequence by year in accession numbers available at PubMed as queried by applicants.

2009 ACU30143 pos 37; 2013 AGW50714 pos 61
(SEQ ID NO: 1)
KLDDLHVAMK
2009 ACU30143 pos 72; AGW50714 pos 96
(SEQ ID NO: 2)
KNDHLSEMVEK
2009 ACU30143 pos 251; 2013 AGW50714 pos 275
(SEQ ID NO: 3)
KQNLKLLETKH
2009 ACU30143 pos 255; 2013 AGW50714 pos 279
(SEQ ID NO: 4)
KLLETKHITEK
2009 ACU30143 pos 506; 2013 AGW50714 pos 530
(SEQ ID NO: 5)
KNYIHIARNLK
2009 ACU30143 pos 367; 2013 AGW50714 pos 391
(SEQ ID NO: 6)
KKAPEDKRTQMH
2009 ACU30143 pos 760; 2013 AGW50714 pos 784
(SEQ ID NO: 7)
KLKLKHRK
2009 ACU30143 pos 881; 2013 AGW50714 pos 905
(SEQ ID NO: 8)
KEHYAFK
2009 ACU30143 pos 862; 2013 AGW50714 pos 886
(SEQ ID NO: 9)
KEGVVKLKLNH
2009 ACU30143 pos 1221; 2013 AGW50714 pos 1245
(SEQ ID NO: 10)
KGLVMPIACHK
2009 ACU30143 pos 1303; 2013 AGW50714 pos 1327
(SEQ ID NO: 11)
KRVTDIEFKGPRH
2009 ACU30143 pos 1311; 2013 AGW50714 pos 1335
(SEQ ID NO: 12)
KGPRHTSVWK
2009 ACU30143 pos 1349; 2013 AGW50714 pos 1373
(SEQ ID NO: 13)
KWYHRFCDIQK
2009 ACU30143 pos 1389; 2013 AGW50714 pos 1413
(SEQ ID NO: 14)
HLFTLLIKDTVDNRK
2009 ACU30143 pos 1132; 2013 AGW50714 pos 1156
(SEQ ID NO: 15)
KQTITDWQKLTH
2009 ACU30143 pos 947; 2013 AGW50714 pos 971
(SEQ ID NO: 16)
HCNNPDNDKYEPLFK
2009 ACU30143 pos 762; 2013 AGW50714 pos 786
(SEQ ID NO: 17)
KLKHRKYK
2009 ACU30143 pos 59; 2013 AGW50714 pos 83
(SEQ ID NO: 18)
KFNWTNVEKLTYEKNDH
2009 ACU30143 pos 42; 2013 AGW50714 pos 66
(SEQ ID NO: 19)
HVAMKFEDLKLAIIDDPK

Because these sequences are conserved in years of high incidence of CCD in Varroa destructor, which is associated with CCD, and because these sequences are included in the Replikin concentrations analysis disclosed herein (which shows coincidence in Replikin sequences in various factors in CCD thereby demonstrating the Lethal Replikins Gene shared among these factors) Replikin sequences identified as conserved in Varro destructor are particularly useful as targets for controlling CCD. Functional fragments and homologues of these conserved sequences are likely useful. As a result, a vaccine may be designed using any one or more of SEQ ID NO(s): 1-19 and may be designed using any one or more homologues of SEQ ID NO(s): 1-19 and may be designed using any one or more functional fragments of SEQ ID NO(s): 1-19 or combination of any of these targets. Because these Replikin sequences are associated with CCD and rapid replication in the pathogens and parasites involved in CCD, targeting of these Replikin sequences and/or their homologues or functional fragments provides methods of control of CCD.

### Example 4

### Synthetic Replikin Vaccine Against Varroa destructor in CCD

A synthetic vaccine was designed against CCD. The vaccine comprises solid-phase synthesized peptides of SEQ ID NO(s): 1-19 in approximately equal parts by weight. The peptides are combined in sterile water and sugar is added to the mixture. The vaccine is presented to a plurality of colonies of bees for consumption. The colonies are monitored for CCD. The vaccine provides reduction in incidence of CCD in colonies to which the vaccine is presented.

### Example 5

### Replikin Sequences in ATPase of Nosema

Applicants analyzed genomic and proteomic information publicly available for amino acid sequences of ATPase isolated from Nosema species at the website of the NCBI. The following Replikin sequences were identified in accession number XP_002996292:

(SEQ ID NO: 20)
HLKMLYTKK;
(SEQ ID NO: 21)
KKRDDHLK;
and
SEQ ID NO: 22)
KRDDHLK

Because these sequences are present in ATPase of Nosema species, which is associated with CCD, and because these sequences are included in the Replikin concentrations analysis disclosed herein (which shows coincidence in Replikin sequences in various factors in CCD thereby demonstrating the Lethal Replikins Gene shared among these factors) Replikin sequences identified in Nosema species are particularly useful as targets for controlling CCD. Functional fragments and homologues of these conserved sequences are likely useful. As a result, a vaccine may be designed using any one or more of SEQ ID NO(s): 20-22 and may be designed using any one or more homologues of SEQ ID NO(s): 20-22 and may be designed using any one or more functional fragments of SEQ ID NO(s): 20-22 or combination of any of these targets. Because these Replikin sequences are associated with CCD and rapid replication in the pathogens and parasites involved in CCD, targeting of these Replikin sequences and/or their homologues or functional fragments provides methods of control of CCD.

### Example 6

### Synthetic Replikin Vaccine Against Nosema Infection in Bees

A synthetic vaccine was designed against CCD. The vaccine comprises solid-phase synthesized peptides of HLKMLYTKK (SEQ ID NO: 20), KKRDDHLK (SEQID NO: 21), and KRDDHLK SEQ ID NO: 22) in approximately equal parts by weight. The peptides are combined in sterile water and sugar is added to the mixture. The vaccine is presented to a plurality of colonies of bees for consumption. The colonies are monitored for CCD.

### Example 7

### Replikin Sequences in ATPase of Honeybees

Applicants analyzed genomic and proteomic information publicly available for ATPase amino acid sequences isolated from honeybees and publicly available at the website of NCBI. The following Replikin sequences were identified as conserved in accession numbers AGC51679 and AGC51702: KFHRVSTK (SEQ ID NO: 23); KIDRWFLHK (SEQ ID NO: 24); KLSHDVLLRAK (SEQ ID NO: 25); KLSHDVLLGAK (SEQ ID NO: 26); KLSHDVLLHAK (SEQ ID NO: 27); KIPRWDLGKFH (SEQ ID NO: 28); HTKLSHDVLLRAK (SEQ ID NO: 29); and HAKRIGFSDK (SEQ ID NO: 30).

Because these sequences are conserved in years of high incidence of CCD in honeybees and because these sequences are included in the Replikin concentrations analysis disclosed herein (which shows coincidence in Replikin sequences in various factors in CCD and honeybee ATPase thereby demonstrating the Lethal Replikins Gene shared among these factors and honeybees themselves) Replikin sequences identified as conserved in ATPase in honeybees are particularly useful as targets for controlling CCD. Functional fragments and homologues of these conserved sequences are likely useful. As a result, a vaccine may be designed using any one or more of SEQ ID NO(s): 23-30 and may be designed using any one or more homologues of SEQ ID NO(s): 23-30 and may be designed using any one or more functional fragments of SEQ ID NO(s): 23-30 or combination of any of these targets. Because these Replikin sequences are associated with CCD and rapid replication in the pathogens and parasites involved in CCD, targeting of these Replikin sequences and/or their homologues or functional fragments provides methods of control of CCD.

### Example 8

### Synthetic Replikin Vaccine Against CCD Using Replikin Sequences in ATPase in Honeybees

A synthetic vaccine was designed against CCD employing Replikin sequences conserved in ATPase in honeybees. The vaccine comprises solid-phase synthesized peptides of SEQ ID NO(s): 23-30 in approximately equal parts by weight. The peptides are combined in sterile water and sugar is added to the mixture. The vaccine is presented to a plurality of colonies of bees for consumption. The colonies are monitored for CCD.

### Example 9

### Replikin Concentrations Identified in Pathogens from the Honeybee Microbiome

Applicants reviewed amino acid sequences disclosed at NCBI in the following accession numbers from Runckel, C. et al., “Temporal analysis of the honeybee microbiome reveals four novel viruses and seasonal prevalence of known viruses, nosema, and crithidia,” JOURNAL PLoS ONE 6 (6), E20656 (2011). Each accession number is disclosed in Table 1 below along with the concentration of Replikins per 100 amino acid residues within each disclosed sequence, the year of publication, and the pathogen from which the sequence was isolated (and the strain, as reported). Replikin sequence identified in these accession numbers are available for targets for control of CCD.

TABLE 1

| | Replikin | | | |
|---|---|---|---|---|
| Accession | concen- | | | |
| No. | tration | Year | Pathogen | Strain |
| AEH42818 | 11.1 | 2009 | Crithidia | BruceSD_T17 |
| | | | mellificae | |
| AEH42817 | 0.6 | 2009 | Big Sioux | BruceSD_T17 |
| | | | River virus | |
| AEH42816 | 0.8 | 2009 | Big Sioux | BruceSD_T17 |
| | | | River virus | |
| AEH42814 | 3.5 | 2009 | Big Sioux | BruceSD_T17 |
| | | | River virus | |
| AEH42813 | 0.8 | 2009 | Big Sioux | BruceSD_T17 |
| | | | River virus | |
| AEH26194 | 1.2 | 2009 | Lake Sinai virus 1 | BruceSD_T17E02 |
| AEH26193 | 1 | 2009 | Lake Sinai virus 1 | BruceSD_T17E02 |
| AEH26192 | 0.8 | 2009 | Lake Sinai virus 1 | BruceSD_T17E02 |
| AEH26191 | 2 | 2009 | Aphid lethal | brookings |
| | | | paralysis virus | |
| AEH26189 | 1 | 2009 | Lake Sinai virus 2 | BruceSD_T17E01 |
| AEH26188 | 1 | 2009 | Lake Sinai virus 2 | BruceSD_T17E01 |
| AEH26187 | 0.6 | 2009 | Lake Sinai virus 2 | BruceSD_T17E01 |

### Example 10

### Replikin Concentrations Identified in ATPase of Honeybees

Applicants reviewed amino acid sequences disclosed at NCBI for ATPase in honeybees. Table 2 below provides mean Replikin concentration, standard deviation, and significance for isolates from the listed years.

TABLE 2

| | | Mean | | |
|---|---|---|---|---|
| | No. of | Replikin | | |
| | Isolates | Count per | | |
| Year | per year | year | S.D | Significance |
| 1993 | 3 | 0.4 | 0.0 | low p < .001 |
| 1994 | 1 | 1.0 | 0.0 | prev p < .001 |
| 1996 | 1 | 0.8 | | |
| 2003 | 1 | 0.6 | | |
| 2004 | 82 | 4.9 | 3.6 | low p < .001, prev p < .0 |
| 2005 | 36 | 5.5 | 4.0 | low p < .001, prev p < .40 |
| 2006 | 109 | 5.4 | 2.4 | low p < .001, prev p > .50 |
| 2007 | 18 | 1.7 | 2.2 | low p < .20, prev p < .001 |
| 2008 | 191 | 2.7 | 2.4 | low p < .001, prev p < .05 |
| 2009 | 86 | 3.7 | 3.6 | low p < .001, prev p < .02 |
| 2010 | 206 | 2.0 | 2.7 | low p < .001, prev p < .001 |
| 2011 | 637 | 4.3 | 3.3 | low p < .001, prev p < .001 |
| 2012 | 520 | 4.3 | 3.3 | low p < .001, prev p > .50 |
| 2013 | 313 | 4.1 | 3.5 | low p < .001, prev p < .40 |

### Example 11

### Replikin Concentrations Identified in Complementary Sex-Determination (csd) Gene of Honeybees

Applicants reviewed amino acid sequences disclosed at NCBI for complementary sex-determination genes (csd) in honeybees. The mean Replikin concentration data is illustrated in FIG. 14.

### Example 12

### Replikin Concentrations Identified in Accession Numbers Reporting Complementary Sex-Determination (csd) Gene of Honeybees and Percent of Isolates with Replikin Concentration Greater than 4.0

Applicants reviewed amino acid sequences disclosed at NCBI for complementary sex-determination genes (csd) in honeybees. Percent of isolates with Replikin concentration above 4.0 was calculated for the periods of 2003 through 2005 and 2006 through 2009 as well as for years 2010, 2011, 2012, and 2013. Data on percent of isolates with Replikin concentration above 4.0 are illustrated in FIG. 15. From 2003 through 2005, Applicants identified thirty-eight isolates. Of those thirty-eight isolates, twenty-seven were observed to have a Replikin concentration of greater than 4.0, which is 71.10% of isolates observed with a Replikin concentration of greater than 4.0. From 2006 through 2009, Applicants identified 192 isolates. Of those 192 isolates, 106 isolates were observed to have a Replikin concentration of greater than 4.0, which is 55.20% of isolates observed with a Replikin concentration of greater than 4.0. Applicants identified twenty-four isolates from 2010. Six of those isolates were observed to have a Replikin concentration of greater than 4.0, which is 25% of isolates observed with a Replikin concentration of greater than 4.0. Applicants identified 193 isolates from 2011. 114 isolates were observed to have a Replikin concentration of greater than 4.0, which is 59.10% of isolates observed with a Replikin concentration of greater than 4.0. Applicants identified 96 isolates from 2012. 45 isolates were observed to have a Replikin concentration of greater than 4.0, which is 46.90% of isolates observed with a Replikin concentration of greater than 4.0. For 2013, Applicants identified 13 isolates through from January to November. Of those 13 isolates, 12 isolates were observed to have a Replikin concentration of greater than 4.0, which is 92.30% of isolates observed with a Replikin concentration of greater than 4.0.

### Example 13

### Replikin Concentrations Identified in 2012 in Csd Gene of Apis Laborious (Himalayan Honeybee)

Applicants reviewed amino acid sequences disclosed at NCBI for Apis laborious (Himalayan honeybee) for Replikin concentration in 2012. Table 3 provides a list of accession numbers with Replikin concentration, year, and source. Each accession number was defined as “Nucleotide Diversity Based on csd Gene of the Black Giant Honey Bee (Hymenoptera: Apidae: A. laboriosa) Direct Submission.”

TABLE 3

| ACCESSION | REPLIKIN | | |
|---|---|---|---|
| NUMBER | COUNT | YEAR | SOURCE |
| AFS41786 | 8 | 2012 | Apis laboriosa |
| | | | (Himalayan honeybee) |
| AFS41785 | 7.5 | 2012 | Apis laboriosa |
| | | | (Himalayan honeybee) |
| AFS41784 | 8.4 | 2012 | Apis laboriosa |
| | | | (Himalayan honeybee) |
| AFS41783 | 8.4 | 2012 | Apis laboriosa |
| | | | (Himalayan honeybee) |
| AFS41782 | 13.2 | 2012 | Apis laboriosa |
| | | | (Himalayan honeybee) |
| AFS41781 | 8 | 2012 | Apis laboriosa |
| | | | (Himalayan honeybee) |
| AFS41780 | 7.5 | 2012 | Apis laboriosa |
| | | | (Himalayan honeybee) |
| AFS41779 | 8 | 2012 | Apis laboriosa |
| | | | (Himalayan honeybee) |
| AFS41778 | 9.6 | 2012 | Apis laboriosa |
| | | | (Himalayan honeybee) |
| AFS41777 | 11.5 | 2012 | Apis laboriosa |
| | | | (Himalayan honeybee) |
| AFS41776 | 10.9 | 2012 | Apis laboriosa |
| | | | (Himalayan honeybee) |
| AFS41775 | 6.3 | 2012 | Apis laboriosa |
| | | | (Himalayan honeybee) |
| AFS41774 | 6.3 | 2012 | Apis laboriosa |
| | | | (Himalayan honeybee) |

### Example 14

### Sequences Conserved by Year in Complementary Sex-Determination Gene in Honeybees

Applicants reviewed amino acid sequences of the complementary sex-determination gene (csd) of honeybees disclosed at NCBI for conserved Replikin sequences. Following is a list of identified conserved Replikin sequences in csd with years in which isolates were identified as having the conserved sequence.

Conserved Replikin sequences are ideal sequences for targeting CCD through targeting rapid replication of disease or parasitic organism in CCD.

2006, 2008, 2011, 2012
(SEQ ID NO: 31)
KHNHYNK
2008, 2012
(SEQ ID NO: 32)
KHYNHYNHYNK
2006, 2008, 2011, 2012
(SEQ ID NO: 33)
KHYNHYNK
2012
(SEQ ID NO: 34)
KHYNNYNNK
2008, 2012
(SEQ ID NO: 35)
KHYNNKHYK
2003, 2004, 2005, 2006, 2007, 2008, 2011,
2012, 2013
(SEQ ID NO: 36)
KLHNEKEK
2008, 2011, 2012
(SEQ ID NO: 37)
HNEKEKFLQEK
2008, 2011, 2012
(SEQ ID NO: 38)
KEKFLQEKTSH
2008, 2011, 2012
(SEQ ID NO: 39)
KFLQEKTSHK
2006, 2008, 2011, 2012
(SEQ ID NO: 40)
KLLQEKTSRKRYSRSREREQKSH
2006, 2008, 2012
(SEQ ID NO: 41)
KHYNNKHYNK
2006, 2008, 2011, 2012
(SEQ ID NO: 42)
KHYNKHYNK
2006, 2008, 2011, 2012
(SEQ ID NO: 43)
KHYNKHYK

SEQ ID NO(s): 31-43, functional fragments thereof, and homologues thereto are provided herein as targets for control of CCD and pathogens and parasites associated with CCD. A non-limiting vaccine may comprise any one or more of the sequences, fragments, or homologues. A vaccine may likewise comprise a mixture of at least each of the sequences. Further, Replikin sequences from other factors in CCD may be combined with any one or more of the sequences, fragment, or homologues.

1. An isolated or chemically-synthesized peptide of up to 100 amino acid residues comprising at least one sequence that is at least 80% homologous with at least one Replikin peptide sequence identified in an isolate of Varroa destructor, Nosema species, Deformed wing virus, Israeli acute paralysis virus, Kashmir bee virus, Sacbrood virus, or in the ATPase or complementary sex-determination gene of a honeybee.

2. The isolated or chemically-synthesized peptide of claim 1 wherein said honeybee is Apis mellifera or Apis cerana.

3. The isolated or chemically-synthesized peptide of claim 1, consisting essentially of said at least one Replikin peptide sequence or a homologue of said at least one Replikin peptide sequence that is 80% homologous with said Replikin peptide sequence.

4. The isolated or chemically-synthesized peptide of claim 1, consisting of said at least one Replikin peptide sequence.

5. The isolated or chemically-synthesized peptide of claim 1, consisting of a functional fragment of said at least one Replikin peptide sequence.

6. The isolated or chemically-synthesized peptide of claim 1, comprising at least one of SEQ ID NO(s): 1-19, 20-22, 23-30, or 31-43.

7. The isolated or chemically-synthesized peptide of claim 1, comprising at least one homologue of at least one of SEQ ID NO(s): 1-19, 20-22, 23-30, or 31-43.

8. A blocking composition comprising at least one isolated or chemically-synthesized peptide of claim 1.

9. The blocking composition of claim 8 comprising a mixture of isolated or chemically-synthesized peptides of each of SEQ ID NO(s): 1-19, SEQ ID NO(s): 20-22, SEQ ID NO(s): 23-30, or SEQ ID NO(s): 31-43.

10. The blocking composition of claim 8 comprising each of the isolated or chemically-synthesized peptides of SEQ ID NO(s): 1-43.

11. The blocking composition of claim 9, wherein said mixture comprises an approximately equal molar mixture of the isolated or synthesized peptides of SEQ ID NO(s): 1-19, 20-22, 23-30, or 31-43.

12. The blocking composition of claim 9, wherein said mixture comprises approximately equal weight of the isolated or synthesized peptides of SEQ ID NO(s): 1-19, 20-22, 23-30, or 31-43.

13-18. (canceled)

19. A binding agent that binds at least a portion of at least one Replikin peptide sequence identified in an isolate of Varroa destructor, Nosema species, Deformed wing virus, Israeli acute paralysis virus, Kashmir bee virus, Sacbrood virus, or identified in the ATPase or complementary sex-determination gene of a honeybee or at least one homologue of said at least one Replikin peptide sequence that is at least 80% homologous with said at least one Replikin peptide sequence.

20. The binding agent of claim 19 that binds at least a portion of at least one of SEQ ID NO(s): 1-43 or a homologue of SEQ ID NO(s): 1-43 that is at least 80% homologous with at least one of SEQ ID NO(s): 1-43.

21-24. (canceled)

25. A method of determining an increased probability of an increase of CCD within about one year following an increase in Replikin concentration in an isolate of a CCD factor comprising identifying an increase in the concentration of Replikin sequences in at least one first isolate of a CCD factor as compared to at least one other isolate of a CCD factor wherein said at least one first isolate is isolated at a later time period than said one other isolate and wherein said increase in the concentration of Replikin sequences signifies the increased probability of the outbreak of a CCD factor within about one year following the increase in the concentration of Replikin sequences.
